(19) United States
(12) Patent Application Publication
(10) Pub. No.: US 2009/0029456 A1
(43) Pub. Date: Jan. 29, 2009

(54) STR50 and uses thereof

(76) Inventors: Sylvia G. Kachalsky, Gan Yavne (IL); Alexander Faerman, Haellah (IL); Yehuda Per-Or, Tel Aviv (IL)

Correspondence Address: COOPER & DUNHAM, LLP, 30 Rockefeller Plaza, 20th Floor, NEW YORK, NY, 10112, US

(21) Appl. No.: 12/151,774

(22) Filed: May 8, 2008

Related U.S. Application Data
(63) Continuation of application No. 10/612,318, filed on Jul. 1, 2003, now abandoned.
(60) Provisional application No. 60/393,251, filed on Jul. 1, 2002.

(51) Int. Cl. C12N 5/06; C07H 21/00; C07K 16/18; C07K 14/435; C12N 15/64

(52) U.S. Cl. 435/325; 536/23.5; 530/412; 530/387.1; 435/320.1

(57) ABSTRACT

The present invention relates to use of a novel gene, STR50 and to STR50 modulators in diagnostic, therapeutic and screening aspects related to neurotoxic events. More particularly, the present invention concerns diagnosing and treating neurodegenerative diseases, and screening for species which can be useful in treating the same.

### PRIORITY

This application is a continuation of U.S. Ser. No. 10/612,318, filed Jul. 1, 2003, which claims benefit of U.S. provisional application No. 60/393,251, filed Jul. 1, 2002, all of which are hereby incorporated by reference in their entirety into this application.

### FIELD OF THE INVENTION

The present invention is related to novel genes and polypeptides, and their relation to neurotoxic events, to diagnosis and treatment of tumors and neurodegenerative diseases, and to screening for modulators of such diseases.

### BACKGROUND OF THE INVENTION

### Ischemia of the Brain

Brain injury such as trauma and stroke are among the leading causes of mortality and disability in the western world.

Traumatic brain injury (TBI) is one of the most serious reasons for hospital admission and disability in modern society. Clinical experience suggests that TBI may be classified into primary damage occurring immediately after injury, and secondary damage, which occurs during several days post injury. Current therapy of TBI is either surgical or else mainly symptomatic.

Cerebrovascular diseases occur predominately in the middle and late years of life. They cause approximately 200,000 deaths in the United States each year as well as considerable neurologic disability. The incidence of stroke increases with age and affects many elderly people, a rapidly growing segment of the population. These diseases cause either ischemia-infarction or intracranial hemorrhage.

Stroke is an acute neurologic injury occurring as a result of interrupted blood supply, resulting in an insult to the brain. Most cerebrovascular diseases present as the abrupt onset of focal neurologic deficit. The deficit may remain fixed, it may improve or progressively worsen, leading usually to irreversible neuronal damage at the core of the ischemic focus, whereas neuronal dysfunction in the penumbra may be treatable and or reversible. Prolonged periods of ischemia result in frank tissue necrosis. Cerebral edema follows and progresses over the subsequent 2 to 4 days. If the region of the infarction is large, the edema may produce considerable mass effect with all of its attendant consequences.

Neuroprotective drugs are being developed in an effort to rescue neurons in the penumbra from dying, though as yet none has been proven efficacious. Damage to neuronal tissue can lead to severe disability and death. The extent of the damage is primarily affected by the location and extent of the injured tissue. Endogenous cascades activated in response to the acute insult play a role in the functional outcome. Efforts to minimize, limit and/or reverse the damage have the great potential of alleviating the clinical consequences.

### SUMMARY OF THE INVENTION

The present invention provides novel genes, polypeptides and antibodies useful in the modulation of ischemic or neurotoxic events; the present invention further provides compositions comprising the novel genes and/or polypeptides, methods for identifying species which modulate the biological activity of these novel genes and/or polypeptides, and methods for diagnosing neurotoxic stress and neurodegenerative diseases.

The preferred methods, materials, and examples that will now be described are illustrative only and are not intended to be limiting; materials and methods similar or equivalent to those described herein can be used in practice or testing of the invention. Other features and advantages of the invention will be apparent from the following detailed description, and from the claims.

### DETAILED DESCRIPTION OF THE INVENTION

The present invention provides novel polynucleotides and polypeptides, small molecules, compositions and methods for diagnosing and alleviating or reducing the symptoms and signs associated with neurotoxic stress or neurodegenerative diseases.

The present invention further provides for screening systems aimed at isolating species which modulate the biological activity of the novel polypeptide disclosed herein, so as to thereby modulate a neurotoxic condition or neurodegenerative disease.

The inventors of the present invention discovered that the expression of the novel gene STR50 is modulated as a result of neurotoxic stress.

The term “apoptosis” is particularly defined as execution of built-in cell death program resulting in chromatin fragmentation into membrane-bound particles, changes in cell cytoskeleton and membrane structure and subsequent phagocytosis of apoptotic cell by other cells. However, as used herein, it should be understood that this term should be construed more broadly as encompassing neuronal cell death, whether or not that cell death is strictly by means of the apoptotic process described above

Novel Gene which Modulates Neurotoxic Events

The present invention provides a novel gene, named STR50_E1 and referred to herein as “STR50”, which can be alternatively spliced and thus exists in two variants; the expression of this novel gene is modulated during stroke. Thus, this novel gene encodes a polypeptide which has the biological activity of, inter alia, modulating neurotoxic events.

Genbank ID No: gi 14248494 discloses a cDNA which has certain similarities to the claimed novel gene. However, the gene sequence disclosed is not complete. The truncated gene sequence lacks the coding sequence for many amino acids at the N-terminus of the polypeptide. To be specific, it lacks the coding sequence for amino acids 1-58 or 1-93 of the short (FIG. 4) and long (FIG. 2) variants respectively of the polypeptide, and therefore does not encode the full polypeptide of either variant of the instant invention.

The term “polynucleotide” refers to any molecule composed of DNA nucleotides, RNA nucleotides or a combination of both types, i.e. that comprises two or more of the bases guanidine, citosine, timidine, adenine, uracil or inosine, inter alia. A polynucleotide may include natural nucleotides, chemically modified nucleotides and synthetic nucleotides, or chemical analogs thereof. The term encompasses “oligonucleotides” and “nucleic acids”. A polynucleotide generally has from about 10 to 10,000 nucleotides, optionally from about 100 to 3,500 nucleotides. An oligonucleotide refers generally to a chain of nucleotides extending from 2-500 nucleotides.

The term “amino acid” refers to a molecule which consists of any one of the 20 naturally occurring amino acids, amino acids which have been chemically modified (see below), or synthetic amino acids.

By “polypeptide” is meant a molecule composed of amino acids and the term includes peptides, polypeptides, proteins and peptidomimetics.

A “peptidomimetic” is a compound containing non-peptidic structural elements that is capable of mimicking the biological action(s) of a natural parent peptide. Some of the classical peptide characteristics such as enzymatically scissille peptidic bonds are normally not present in a peptidomimetic.

The term “STR50”, as used herein, also known as “MEG-3”, refers to the expressed polypeptide of either variant of the novel gene STR50, derived from any organism, preferably man, and splice variants thereof. In addition, this term is understood to encompass polypeptides resulting from minor alterations in the coding sequence of either variant of the STR50 gene, such as, inter alia, point mutations, substitutions, deletions and insertions which may cause a difference in a few amino acids between the resultant polypeptide and the naturally occurring STR50 polypeptides. Polypeptides encoded by nucleic acid sequences which bind to the STR50 coding sequences or genomic sequence under conditions of highly stringent hybridization, which are well-known in the art (for example Ausubel et al., Current Protocols in Molecular Biology, John Wiley and Sons, Baltimore, Md. (1988), updated in 1995 and 1998), are also encompassed by this term. Chemically modified STR50 polypeptides or chemically modified fragments of the STR50 polypeptide are also included in the term, so long as the biological activity is retained. The cDNA sequence (including untranslated regions) and amino acid sequence of the long variant of STR50 are set out in FIGS. 1A-1C (SEQ ID NO:1) and 2 (SEQ ID NO:2) respectively; The cDNA sequence (including untranslated regions) and amino acid sequence of the short variant of STR50 are set out in FIGS. 3A-3C (SEQ ID NO:3) and 4 (SEQ ID NO:4) respectively. Additionally, molecules comprising the N-terminus of the polypeptides encoded by the sequences set forth in FIG. 2 (SEQ ID NO:2) and FIG. 4 (SEQ ID NO:4) are also encompassed by this term, so long as they retain STR50 biological activity. The “N-terminus” as used herein typically refers to 10 consecutive amino acids starting from position 1 of the amino acid sequence (i.e., SEQ ID NO:2 or SEQ ID NO:4).

Further, homologs and fragments of STR50 having similar biological activity and homologs thereof (including the murine homolog) having at least 50%, preferably 60% or 70%, more preferably at least 80%, even more preferably at least 90% or 95% homology thereto are envisaged, so long as they are not disclosed in Genbank ID No. 14248494.

Particular fragments of STR50 include amino acids 1-50, 51-100, 101-150, 151-200, 201-250, 251-300, 301-350, 351-400, 401-450, 451-500, 501-550, 551-600, 601-650, 651-700, 701-750, 751-800, and 801-821 of the sequence shown in FIG. 2 (SEQ ID NO:2). Further particular fragments of STR50 include amino acids 25-74, 75-124, 125-174, 175-224, 225-274, 275-324, 325-374, 375-424, 425-474, 475-524, 525-574, 575-624, 625-674, 675-724, 725-774 and 775-821 of the sequence shown in FIG. 2 (SEQ ID NO:2).

The present invention provides a purified STR50 polypeptide, optionally comprising consecutive amino acids the sequence of which extends from position 1 through position 821 of SEQ ID No:2, further optionally comprising consecutive amino acids the sequence of which extends from position 1 through position 791 of SEQ ID No:4. Further, the present invention provides a purified polypeptide encoded by a polynucleotide having between 10 and 922 consecutive nucleotides from position 1 to position 922 of SEQ ID NO:1 or by a polynucleotide having between 10 and 766 consecutive nucleotides from position 1 to position 766 of SEQ ID NO:3. Further, said polypeptides as described above may have the biological activity of modulating neurotoxic stress; in this respect, the present invention further comprises a purified polypeptide having at least 70% homology to, and retaining said biological activity.

By “STR50 biological activity” or “activity of STR50” is meant the activity of STR50 in neurotoxic events. The inventors of the present invention have found that the expression of STR50 rises as a result of such neurotoxic events. Without being bound by theory, the inventors suggest that STR50 is involved in the apoptosis of cells which accompanies neurotoxic events; it would therefore be beneficial to inhibit STR50 in diseases where such apoptosis is detrimental, and enhance STR50 in diseases where such apoptosis is beneficial.

The term “neurotoxic stress” as used herein is intended to comprehend any stress that is toxic to normal neural cells (and may cause their death or apoptosis). Such stress may be oxidative stress, hypoxia, hyperoxia, ischemia or trauma, and/or it may involve subjecting the cells to a substance that is toxic to the cells in vivo, such as glutamate or dopamine or the Aβ protein, or any substance or treatment that causes oxidative stress. The neurotoxic substance may be endogenous or exogenous and the term neurotoxic is also intended to comprehend exposure to various known neurotoxins including organophosphorous poisoning, or any other insult of this type.

The term “neurotoxic event” refers to any event in which any type of neurotoxic stress is involved.

Additionally, the present invention provides a purified polypeptide comprising consecutive amino acids the sequence of which extends from the position of any one of the Methionine amino acids present in the sequence, or the amino acid immediately following the Methionine, through to position 821 of SEQ ID No:2 depicted in FIG. 2. In addition and as described herein, since the novel gene STR50 has two splice variants of the expressed polypeptide, the present invention further provides for a purified polypeptide comprising consecutive amino acids the sequence of which extends from position 63 through position 791 of SEQ ID No:4, or from position 62 through position 791 of SEQ ID No:4, or from position 60 through position 791 of SEQ ID No:4, or from position 59 through position 791 of SEQ ID No:4, or from position 2 through position 791 of SEQ ID No:4, or from position 1 through position 791 of SEQ ID No:4. SEQ ID No:4 is depicted in FIG. 4.

The present invention further provides a purified polypeptide encoded by a polynucleotide having at least 15, optionally at least 30, 50, 70 or even 100 consecutive nucleotides from position 1 to position 922 of the sequence depicted in FIGS. 1A-1C. In addition, the present invention provides a purified polypeptide encoded by a polynucleotide having at least 15, optionally at least 30, 50, 70 or even 100 consecutive nucleotides from position 1 to position 766 of the sequence depicted in FIGS. 3A-3C.

Any purified polypeptides encoded by polynucleotides of at least 15 consecutive nucleotides depicted in either FIGS. 1A-1C or FIGS. 3A-3C are considered to be a part of the present invention, provided that they are not disclosed in Genbank ID number gi: 14248494; these polynucleotides are also considered a part of the present invention.

An additional embodiment of the present invention provides for the above polypeptides having the biological activity of modulating neurotoxic stress, and any polypeptide which is at least 70% homologous thereto and retains the biological activity thereof.

The present invention further provides an antibody that binds to an epitope on STR50, in any of its variants and different possible fragments or altered polypeptides, i.e., an antibody binding specifically to any of the above polypeptides, all as described herein. Specifically, the antibody may bind to an epitope on a polypeptide comprising consecutive amino acids the sequence of which extends from position 1 through position 821 of SEQ ID No:2, a polypeptide comprising consecutive amino acids the sequence of which extends from position 1 through position 791 of SEQ ID No:4, a polypeptide encoded by a polynucleotide having between 10 and 922 consecutive nucleotides from position 1 to position 922 of SEQ ID NO:1, or a polypeptide encoded by a polynucleotide having between 10 and 766 consecutive nucleotides from position 1 to position 766 of SEQ ID NO:3. In addition, the present invention provides for an antibody which binds to an epitope on a polypeptide having at least 70% homology to, and retaining the biological activity of STR50. Such antibodies may not bind to a polypeptide encoded by the cDNA disclosed in Genbank ID No. gi 14248494 (see above).

The term “antibody” as used herein refers to IgG, IgM, IgD, IgA, and IgE antibody, inter alia. The definition includes polyclonal antibodies or monoclonal antibodies. This term refers to whole antibodies or fragments of antibodies, e.g. antibodies without the Fc portion, single chain antibodies, fragments consisting of essentially only the variable, antigen-binding domain of the antibody, etc. The term “antibody” may also refer to antibodies against nucleic acid sequences obtained by cDNA vaccination. The term also encompasses antibody fragments which retain the ability to selectively bind with their antigen or receptor and are exemplified as follows, inter alia:
(1) Fab, the fragment which contains a monovalent antigen-binding fragment of an antibody molecule can be produced by digestion of whole antibody with the enzyme papain to yield a light chain and a portion of the heavy chain;
(2) (Fab′)₂, the fragment of the antibody that can be obtained by treating whole antibody with the enzyme pepsin without subsequent reduction; F(ab′₂) is a dimer of two Fab fragments held together by two disulfide bonds;
(3) Fv, defined as a genetically engineered fragment containing the variable region of the light chain and the variable region of the heavy chain expressed as two chains; and
(4) Single chain antibody (SCA), defined as a genetically engineered molecule containing the variable region of the light chain and the variable region of the heavy chain linked by a suitable polypeptide linker as a genetically fused single chain molecule.

By the term “epitope” as used in this invention is meant an antigenic determinant on an antigen to which the antibody binds. Epitopic determinants usually consist of chemically active surface groupings of molecules such as amino acids or sugar side chains and usually have specific three dimensional structural characteristics, as well as specific charge characteristics.

In an additional embodiment, the present invention concerns a pharmaceutical composition comprising any one of the STR50 polypeptides, as disclosed herein.

Administration of a pharmaceutical composition according to the invention can be carried out by any known route of administration, including intravenously, intra-arterially, subcutaneously, intrathecally or intracerebrally. Using specialized formulations, it may also be possible to administer these orally or via inhalation. Suitable doses and treatment regimens for administering compositions to an individual in need thereof are discussed in detail below.

An additional aspect of the present invention provides a purified polynucleotide which encodes STR50, optionally comprising:

consecutive nucleotides having a sequence as set forth in SEQ ID NO:1 and homologs or complements thereof;
consecutive nucleotides having a sequence as set forth in SEQ ID NO:3 and homologs or complements thereof;
consecutive nucleotides having a sequence as set forth from position 644 through position 3109 of SEQ ID NO:1 and homologs or complements thereof; or
consecutive nucleotides having a sequence as set forth from position 644 through position 3019 of SEQ ID NO:3 and homologs or complements thereof.

Further in this aspect, the invention provides a purified polynucleotide which encodes a polypeptide having the sequence set forth in SEQ ID NO:2, and a purified polynucleotide which encodes a polypeptide having the sequence set forth in SEQ ID NO:4. In addition, a polynucleotide having between 10 and 922 consecutive nucleotides from position 1 to position 922 of SEQ ID NO:1, and a polynucleotide having between 10 and 766 consecutive nucleotides from position 1 to position 766 of SEQ ID NO:3 are also provided. Any polynucleotide comprising at least 15 consecutive nucleotides from the sequence depicted in FIGS. 1A-1C or from the sequence depicted in FIGS. 3A-3C is also considered a part of the present invention, provided that it is not disclosed in Genbank ID number gi: 14248494.

In addition, antisense polynucleotides or fragments to all of the above polynucleotides are also provided.

Further, the present invention provides polynucleotides encoding polypeptides which act as dominant negative peptides to the above polypeptides, and polynucleotides which are siRNAs of the STR50 gene.

By the term “antisense” (AS) or “antisense fragment” is meant a nucleic acid fragment having inhibitory antisense activity, said activity causing a decrease in the expression of the endogenous genomic copy of STR50. The sequence of the AS is designed to complement a target mRNA of interest and form an RNA:AS duplex. This duplex formation can prevent processing, splicing, transport or translation of the relevant mRNA. Moreover, certain AS nucleotide sequences can elicit cellular RNase H activity when hybridized with their target mRNA, resulting in mRNA degradation (Calabretta et al, 1996: Antisense strategies in the treatment of leukemias. Semin Oncol. 23(1):78-87). In that case, RNase H will cleave the RNA component of the duplex and can potentially release the AS to further hybridize with additional molecules of the target RNA. An additional mode of action results from the interaction of AS with genomic DNA to form a triple helix which can be transcriptionally inactive.

The AS fragment of the present invention preferably has the sequence depicted in FIGS. 3A-3C or a homologous sequence thereof. Particular AS fragments are the AS of the DNA encoding the particular fragments of STR50 described above. For delivery of AS fragments see Example 10.

By “silencing RNA” (siRNA) is meant an RNA molecule which decreases or silences the expression of a gene/mRNA of its endogenous or cellular counterpart. The term is understood to encompass “RNA interference” (RNAi), and “double-stranded RNA” (dsRNA). For recent information on these terms and proposed mechanisms, see Bernstein E., Denli A M., Hannon G J: The rest is silence. RNA. 2001 November; 7(11):1509-21; and Nishikura K.: A short primer on RNAi: RNA-directed RNA polymerase acts as a key catalyst. Cell. 2001 Nov. 16; 107(4):415-8.

In addition, the invention provides for a vector comprising any of the above polynucleotides, and a cell comprising said vector. Selection of vectors is well known to those of skill in the art, and will depend on the desired purpose of the clone comprising the desired polynucleotide. For further information see Sambrook et al., Molecular cloning: A laboratory manual, Cold Springs Harbor Laboratory, New-York (1989, 1992), and Ausubel et al., Current Protocols in Molecular Biology, John Wiley and Sons, Baltimore, Md. (1988). Cells appropriate for transformation, transfection, infection or other genetic modifications with suitable vectors are also routinely selected by those of skill in the art, and for the purposes of the present invention may be prokaryotic or eukaryotic cells.

By “STR50 gene” is meant the STR50 coding sequence open reading frame, as shown in FIGS. 1A-1C (SEQ ID NO:1), or any sequences derived from the sequence of FIGS. 1A-1C which have undergone mutations, such as substitutions, deletions and insertions, as described herein, and any nucleic acid that encodes the STR50 polypeptide, as defined herein.

Further, homologous sequence thereof preferably having at least 75% homology, 80% homology or more preferably 90% or 95% homology are envisaged, so long as they are not disclosed in Genbank ID No. 14248494.

Another embodiment of the present invention concerns a pharmaceutical composition comprising any of the above polynucleotides or vectors, and administration of such pharmaceuticals is discussed herein.

By “homolog/homology”, as utilized in the present invention, is meant at least about 70%, preferably at least about 75% homology, advantageously at least about 80% homology, more advantageously at least about 90% homology, even more advantageously at least about 95%, e.g., at least about 97%, about 98%, about 99% or even about 100% homology. The invention also comprehends that these polynucleotides and polypeptides can be used in the same fashion as the herein or aforementioned polynucleotides and polypeptides.

Alternatively or additionally, “homology”, with respect to sequences, can refer to the number of positions with identical nucleotides or amino acid residues, divided by the number of nucleotides or amino acid residues in the shorter of the two sequences, wherein alignment of the two sequences can be determined in accordance with the Wilbur and Lipman algorithm ((1983) Proc. Natl. Acad. Sci. USA 80:726), for instance, using a window size of 20 nucleotides, a word length of 4 nucleotides, and a gap penalty of 4, and computer-assisted analysis and interpretation of the sequence data, including alignment can be conveniently performed using commercially available programs (e.g., Intelligenetics™ Suite, Intelligenetics Inc., CA). When RNA sequences are said to be similar, or to have a degree of sequence identity or homology with DNA sequences, thymidine (T) in the DNA sequence is considered equal to uracil (U) in the RNA sequence. RNA sequences within the scope of the invention can be derived from DNA sequences or their complements, by substituting thymidine (T) in the DNA sequence with uracil (U).

Additionally or alternatively, amino acid sequence similarity or homology can be determined, for instance, using the BlastP program (Altschul et al., Nucl. Acids Res. 25:3389-3402) and available at NCBI. The following references provide algorithms for comparing the relative identity or homology of amino acid residues of two polypeptides, and additionally, or alternatively, with respect to the foregoing, the teachings in these references can be used for determining percent homology: Smith et al., (1981) Adv. Appl. Math. 2:482-489; Smith et al., (1983) Nucl. Acids Res. 11:2205-2220; Devereux et al., (1984) Nucl. Acids Res. 12:387-395; Feng et al., (1987) J. Molec. Evol. 25:351-360; Higgins et al., (1989) CABIOS 5:151-153; and Thompson et al., (1994) Nucl. Acids Res. 22:4673-4680.

In an additional aspect of the present invention, a method of treating a tumor or an auto-immune disease in a subject is provided, which comprises administering to the subject a therapeutically effective amount of a chemical composition which modulates the biological activity of the polypeptide of the present invention. This aspect includes treatment of, inter alia, any disease which involves uncontrolled, pathological cell growth.

This aspect further provides a method of treating neurodegenerative disease in a subject which comprises administering to the subject a therapeutically effective amount of a chemical composition which modulates the biological activity of any one of the polypeptides of the present invention.

The term “neurodegenerative disease” includes, inter alia, stroke, Parkinson's disease, epilepsy, depression, ALS (Amyotrophic lateral sclerosis), Alzheimer's disease, Huntington's disease and any other disease-induced dementia (such as HIV induced dementia for example). In addition, “neurodegenerative disease” is understood to include any conditions which are caused by an ischemic episode and/or which include neurotoxic stress.

It is known in the art, that in certain neurological diseases (for example: brain ischemia or stroke), the blood brain barrier (BBB) is relatively open compared to that of a normal subject, thus enabling penetration of molecules to the brain, even large molecules such as macromolecules, including antibodies, and subsequently allowing interaction of said molecules with STR50.

The treatment regimen according to the invention is carried out, in terms of administration mode, timing of the administration, and dosage, so that the functional recovery of the patient from the adverse consequences is improved; i.e., any one of the patient's motor skills (e.g., posture, balance, grasp, or gait), cognitive skills, speech, and/or sensory perception (including visual ability, taste, olfaction, and proprioception) improve as a result of administration according to the invention.

The invention can be used, inter alia, to treat the adverse consequences of central nervous system injuries that result from any of a variety of conditions. Thrombus, embolus, and systemic hypotension are among the most common causes of cerebral ischemic episodes. Other injuries may be caused by hypertension, hypertensive cerebral vascular disease, rupture of an aneurysm, an angioma, blood dyscrasias, cardiac failure, cardiac arrest, cardiogenic shock, septic shock, head trauma, spinal cord trauma, seizure, bleeding from tumor, or other blood loss.

Where the ischemia is associated with stroke, it can be either global or focal ischemia, as defined below. It is believed that the administration of a pharmaceutical composition according to the invention is effective, even though administration occurs a significant amount of time following the injury.

By “ischemic episode” is meant any circumstance that results in a deficient supply of blood to a tissue. Cerebral ischemic episodes result from a deficiency in the blood supply to the brain. The spinal cord, which is also part of the central nervous system, is equally susceptible to ischemia resulting from diminished blood flow. An ischemic episode may be caused by hypertension, hypertensive cerebral vascular disease, rupture of aneurysm, a constriction or obstruction of a blood vessel (as occurs in the case of a thrombus or embolus), angioma, blood dyscrasias, any form of compromised cardiac function including cardiac arrest or failure, systemic hypotension, cardiac arrest, cardiogenic shock, septic shock, spinal cord trauma, head trauma, seizure, bleeding from a tumor, or other blood loss.

It is expected that the invention will also be useful for treating injuries to the central nervous system that are caused by mechanical forces, such as a blow to the head or spine. Trauma can involve a tissue insult such as an abrasion, incision, contusion, puncture, compression, etc., such as can arise from traumatic contact of a foreign object with any locus of or appurtenant to the head, neck, or vertebral column. Other forms of traumatic injury can arise from constriction or compression of CNS tissue by an inappropriate accumulation of fluid (for example, a blockade or dysfunction of normal cerebrospinal fluid or vitreous humor fluid production, turnover, or volume regulation, or a subdural or intracarnial hematoma or edema). Similarly, traumatic constriction or compression can arise from the presence of a mass of abnormal tissue, such as a metastatic or primary tumor.

By “focal ischemia” as used herein in reference to the central nervous system, is meant the condition that results from the blockage of a single artery that supply blood to the brain or spinal cord, resulting in the death of all cellular elements (pan-necrosis) in the territory supplied by that artery.

By “global ischemia” as used herein in reference to the central nervous system, is meant the condition that results from general diminution of blood flow to the entire brain, forebrain, or spinal cord, which causes the death of neurons in selectively vulnerable regions throughout these tissues. The pathology in each of these cases is quite different, as are the clinical correlates. Models of focal ischemia apply to patients with focal cerebral infarction, while models of global ischemia are analogous to cardiac arrest, and other causes of systemic hypotension.

By “treating a disease” is meant administering a therapeutic substance effective to ameliorate symptoms associated with a disease, to lessen the severity or cure the disease, or to prevent the disease from occurring.

By “effective amount” is meant an amount of a pharmaceutical compound or composition which is effective to achieve an improvement in a patient or his physiological systems including, but not limited to, improved survival rate, more rapid recovery, or improvement or elimination of symptoms, and other indicators as are selected as appropriate determining measures by those skilled in the art.

By “in conjunction with” is meant prior to, simultaneously or subsequent to.

This aspect of the invention may employ, for example, gene therapy. “Gene therapy” means gene supplementation where an additional reference copy of a gene of interest is inserted into a patient's cells. As a result, the polypeptide encoded by the reference gene corrects the defect and permits the cells to function normally, thus alleviating disease symptoms. In the present invention, the reference copy is the STR50 gene and other polynucleotides of the invention which encode the STR50 polypeptide or homologs or fragments thereof that retain the biological activity, and the disease is preferably a degenerative disease, most preferably a neurodegenerative disease.

Gene therapy of the present invention can be carried out in vivo or ex vivo. Ex vivo gene therapy requires the isolation and purification of patient cells, the introduction of a therapeutic gene and the introduction of the genetically altered cells back into the patient.

A replication-deficient virus such as a modified retrovirus can be used to introduce the therapeutic STR50 gene into such cells. For example, mouse Moloney leukemia virus (MMLV) is a well-known vector in clinical gene therapy trials. See, e.g., Boris-Lauerie et al., Curr. Opin. Genet. Dev., 3, 102-109 (1993).

In contrast, in vivo gene therapy does not require isolation and purification of a patient's cells. The therapeutic gene is typically “packaged” for administration to a patient such as in liposomes or in a replication-deficient virus such as adenovirus as described by Berkner, K. L., in Curr. Top. Microbiol. Immunol., 158, 39-66 (1992) or adeno-associated virus (AAV) vectors as described by Muzyczka, N., in Curr. Top. Microbiol. Immunol., 158, 97-129 (1992) and U.S. Pat. No. 5,252,479. Another approach is administration of “naked DNA” in which the therapeutic gene is directly injected into the bloodstream or muscle tissue. Still another approach is administration of “naked DNA” in which the therapeutic gene is introduced into the target tissue by microparticle bombardment using gold particles coated with the DNA. Gene therapy vectors can be delivered to a subject by, for example, intravenous injection, local administration (see U.S. Pat. No. 5,328,470) or by stereotactic injection (see e.g., Chen et al. (1994) PNAS 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g. retroviral vectors, the pharmaceutical preparation can include one or more cells which produce the gene delivery system.

Cell types useful for gene therapy of the present invention include lymphocytes, hepatocytes, myoblasts, fibroblasts, and any cell of the eye such as retinal cells, epithelial and endothelial cells. Preferably the cells are T lymphocytes drawn from the patient to be treated, hepatocytes, any cell of the eye or respiratory or pulmonary epithelial cells. Transfection of pulmonary epithelial cells can occur via inhalation of a neubulized preparation of DNA vectors in liposomes, DNA-protein complexes or replication-deficient adenoviruses. See, e.g., U.S. Pat. No. 5,240,846. For a review of the subject of gene therapy, in general, see the text “Gene Therapy” (Advances in Pharmacology 40, Academic Press, 1997).

A further aspect of the present invention provides for the use of the polypeptides, polynucleotides or antibodies of the present invention in the preparation of a medicament; said medicament may be employed in other aspects of the invention, as described above and herein. Further, said medicament may be employed in the treatment of neurotoxic events, neurotoxic stress, and neurodegenerative diseases.

Another embodiment of the present invention concerns a method for diagnosing a neurodegenerative disease in a subject comprising detecting modulation of the expression level of the polypeptide of the present invention in the subject as compared to a control, whereas said modulation of expression is indicative of the likelihood of neurodegenerative disease in the subject; indeed, the diagnostic methods of the present invention may be practiced on a subject suspected to have undergone a stroke.

The expression level of the polypeptide can be assessed by assaying for mRNA encoding the polypeptide (such as that described in FIGS. 1A-1C or FIGS. 3A-3C, or a fragment or homolog thereof, or by method of an immunoassay using antibodies which detect the polypeptide. Both detection of mRNA and immunoassays can be performed by methods well known in the art (see Examples 2-5 for further details). Measurement of level of the STR50 polypeptide is determined by a method selected from the group consisting of immunohistochemistry (Microscopy, Immunohistochemistry and Antigen Retrieval Methods: For Light and Electron Microscopy, M. A. Hayat (Author), Kluwer Academic Publishers, 2002; Brown C.: “Antigen retrieval methods for immunohistochemistry”, Toxicol Pathol 1998; 26(6): 830-1), western blotting (Laemmeli U K: “Cleavage of structural proteins during the assembley of the head of a bacteriophage T4”, Nature 1970; 227: 680-685; Egger & Bienz, “Protein (western) blotting”, Mol Biotechnol 1994; 1(3): 289-305), ELISA (Onorato et al., “Immunohistochemical and ELISA assays for biomarkers of oxidative stress in aging and disease”, Ann NY Acad Sci 1998 20; 854: 277-90), antibody microarray hybridization (Huang, “detection of multiple proteins in an antibody-based protein microarray system, Immunol Methods 2001 1; 255 (1-2): 1-13) and targeted molecular imaging (Thomas, Targeted Molecular Imaging in Oncology, Kim et al (Eds)., Springer Verlag, 2001).

Measurement of level of STR50 polynucleotide is determined by a method selected from: RT-PCR analysis, in-situ hybridization (“Introduction to Fluorescence In Situ Hybridization: Principles and Clinical Applications”, Andreeff & Pinkel (Editors), John Wiley & Sons Inc., 1999), polynucleotide microarray and Northern blotting (Trayhurn, “Northern blotting”, Proc Nutr Soc 1996; 55(1B): 583-9; Shifman & Stein, “A reliable and sensitive method for non-radioactive Northern blot analysis of nerve growth factor mRNA from brain tissues”, Journal of Neuroscience Methods 1995; 59: 205-208). This diagnostic method may be useful, inter alia, for diagnosing patients suspected to have undergone a stroke.

By “abnormal” or “modulation” in the context of protein expression, is meant a difference of at least 10% in the expression levels of the polypeptide as compared to a control.

An additional aspect of the present invention relates to screening systems, and provides several options of processes for obtaining different types of modulators, which may be inhibitors or enhancers.

In this respect, a cell-based process for obtaining a species which modulates the transcription of a polynucleotide which encodes STR50 is provided, comprising:
(a) measuring the level of transcription of the STR50 gene in a cell;
(b) contacting the cell with a species;
(c) measuring the level of transcription of the STR50 gene in the cell in the presence of the species; and
(d) comparing the level of transcription in (a) to the level of transcription in (c), wherein a modulation in the level of transcription in (c) as compared to the level of transcription in (a) is indicative of the ability of the species to modulate the transcription of a polynucleotide which encodes STR50.

In addition, a non cell-based process for obtaining a species which modulates the translation of a polynucleotide which encodes STR50 is provided comprising:
(a) measuring the level of STR50 in a cell;
(b) contacting the cell with a species;
(c) measuring the level of STR50 in the cell in the presence of the species; and
(d) comparing the level of STR50 in (a) to the level of STR50 in (c), wherein a modulation in the level of STR50 in (c) as compared to the level of STR50 in (a) is indicative of the ability of the species to modulate the transcription of a polynucleotide which encodes STR50.

Further, this aspect of the invention provides a process of preparing a pharmaceutical composition which comprises:
(a) obtaining a species which modulates the transcription or translation of a polynucleotide which encodes STR50, according to the processes described above; and
(b) admixing said species or a chemical analog or homolog thereof with a pharmaceutically acceptable carrier.

An additional embodiment of the screening aspect of the present invention provides a process for obtaining a species which modulates the biological activity of STR50 which comprises:
(a) contacting a cell expressing STR50 with the species; and
(b) determining the ability of the species to modulate the biological activity of STR50 as compared to a control.

The cell in the contacting step may be genetically modified to express STR50. “Genetically modified” in this context includes transduced, transfected, and infected with a polynucleotide or vector which causes the cell to express the polypeptide.

Further, this aspect of the invention provides a process of preparing a pharmaceutical composition which comprises:
(a) obtaining a species according to the above process; and
(b) admixing said species or a chemical analog or homolog thereof with a pharmaceutically acceptable carrier.

In addition, the screening aspect of the present invention provides a process of screening a plurality of species to obtain a species which modulates the biological activity of STR50, (e.g., which modulates neurotoxic stress) which comprises:
(a) contacting cells expressing STR50 with a plurality of species;
(b) determining whether the biological activity of STR50 is modulated in the presence of the species, as compared to a control; and if so
(c) separately determining whether the modulation of the biological activity of STR50 is increased by each species included in the plurality of species, so as to thereby identify the species which modulates the biological activity of STR50.

The cell in the contacting step may be genetically modified to express STR50. Further, this aspect of the invention provides a process of preparing a pharmaceutical composition which comprises:
(a) obtaining a species according to the above process; and
(b) admixing said species or a chemical analog or homolog thereof with a pharmaceutically acceptable carrier.

An additional embodiment of the screening aspect of the present invention provides a non cell-based process for obtaining a species which modulates the biological activity of STR50, comprising:
(a) measuring the interaction of STR50 to an interactor;
(b) contacting STR50 with a species; and
(c) determining whether the interaction between STR50 and the interactor is affected by the species.

Further, this aspect of the invention provides a process of preparing a pharmaceutical composition which comprises:
(a) obtaining a species according to the above process; and
(b) admixing said species or a chemical analog or homolog thereof with a pharmaceutically acceptable carrier.

In a further embodiment of the present invention, a process for obtaining a species which modulates the biological activity of a polypeptide of the present invention is provided which comprises:
(a) contacting a cell expressing a polypeptide of the present invention with the species; and
(b) determining the ability of the species to modulate the biological activity of the polypeptide as compared to a control.

The cell of step (a) may be genetically modified to express the polypeptide. Further, the biological activity of the polypeptide may be that of that of modulating neurotoxic stress, which may be caused by a neurodegenerative disease, such as stroke.

The species may be a chemical compound, a polypeptide, or a polynucleotide; in general, the term “species” encompasses chemical compounds, antibodies, antisense oligonucleotides, antisense DNA or RNA molecules, proteins, polypeptides and peptides including peptido-mimetics and dominant negative peptides, expression vectors and any other molecule capable of interacting with a naturally occurring molecule.

The term “chemical compound” includes the term “small molecule” and “small chemical compound” and is understood to refer to chemical moieties of any particular type which are not necessarily naturally occurring and typically have a molecular weight of less than 2000 daltons, more preferably less than 1000 daltons.

The term “modulation” (i.e., the activity of a modulator) is understood to include partial or full inhibition, stimulation and enhancement.

“Modulator”—any molecule that is capable of modulation, i.e. that either increases (promotes) or decreases (prevents). The term is understood to include partial or full inhibition, stimulation and enhancement. In the case of a modulator of a polypeptide, such as a the STR50 polypeptide, the modulator may be a direct modulator of the biological activity of STR50, or it may be a modulator of the STR50 gene; in the latter case, the activity of STR50 is indirectly modulated by a modulator that affects the transcription or translation of the gene (and does not directly act on the polypeptide). Modulators can include AS fragments, siRNAs, ribozymes, polypeptides and small chemical molecules, inter alia.

“Inhibitor” generally refers to a molecule which is capable of partially or fully inhibiting the biological activity of a gene. In the case of STR50, the term refers to a molecule which partially or fully inhibits STR50 biological activity. Similarly to a modulator, an inhibitor may be a direct inhibitor of the activity of STR50, or it may be an inhibitor of the STR50 gene; in the latter case, the activity of STR50 is indirectly inhibited by an inhibitor that affects the transcription or translation of the gene (and does not directly act on the polypeptide). Examples of different types of inhibitors are, inter alia: nucleic acids such as AS fragments, siRNA, or vectors comprising them; polypeptides such as dominant negatives, antibodies, or, in some cases, enzymes; catalytic RNAs such as ribozymes; and small chemical molecules.

Another embodiment of the present invention concerns a process of screening a plurality of species not known to modulate the biological activity of the polypeptide of the present invention, to obtain a species which modulates the biological activity of a polypeptide of the present invention, which comprises:
(a) contacting cells expressing a polypeptide of the present invention with the plurality of species not known to modulate the biological activity of said polypeptide;
(b) determining whether the biological activity of the polypeptide is modulated in the presence of the species, as compared to a control; and if so
(c) separately determining whether the modulation of the biological activity of the polypeptide is increased by each species included in the plurality of species, so as to thereby identify the species which modulates the biological activity of the polypeptide.

The cell of step (a) may be genetically modified to express the polypeptide. Further, the biological activity of the polypeptide may be that of that of modulating neurotoxic stress, which may be caused by a neurodegenerative disease, such as stroke.

In a further embodiment of the present invention, a non cell-based method for identifying a species which modulates the biological activity of a polypeptide or of a polynucleotide of the present invention is provided, which comprises:
(a) measuring the interaction of a polypeptide or of a polynucleotide of the present invention to an interactor;
(b) contacting the polypeptide or the polynucleotide with said species; and
(c) determining whether the interaction between the polypeptide or the polynucleotide and said interactor is affected by said species.

The term “interactor” refers to any molecule or compound, whether organic or inorganic, which can form an interaction with the specified molecule, be it inter alia a binding interaction, electrostatic interaction, ionic interaction or any other interaction in which the interactor affects the specified molecule in any way.

The present invention further provides a method of preparing a pharmaceutical composition which comprises:
(a) identifying according to the methods described herein a species which modulates the biological activity of the polypeptide or of the polynucleotide of the present invention, and;
(b) admixing said species or a chemical analog or homolog thereof with a pharmaceutically acceptable carrier.

By “chemical analog” as used herein is meant a molecule derived from the originally identified species, that retains the activity, more specifically that retains the activity observed in the parent molecule; chemical analogs may also share structural properties with the parent species.

Additionally, the present invention provides a kit for identifying a species which modulates the biological activity of the polypeptide or of the polynucleotide of the present invention comprising:
(a) the polypeptide or of the polynucleotide of the present invention; and
(b) an interactor with which the polypeptide or the polynucleotide interacts;
(c) means for measuring the interaction of the polypeptide or the polynucleotide with the interactor;
(d) means of contacting the polypeptide or the polynucleotide with said species; and
(e) means of determining whether the interaction between the polypeptide or the polynucleotide and the interactor is affected by said species.

The term “Deletion”—is a change in sequence of either nucleotide or amino acid molecule in which one or more nucleotides or amino acid residues, respectively, are absent.

The term “Insertion” or “addition”—is that change in a sequence of a nucleotide or amino acid molecule resulting in the addition of one or more nucleotides or amino acid residues, respectively, as compared to the naturally occurring molecule.

The term “Substitution” refers to the replacement of one or more nucleotides or amino acids by different nucleotides or amino acids, respectively. As regards amino acid sequences the substitution may be conservative or non-conservative.

The term “Conservative substitution” refers to the substitution of an amino acid in one class by an amino acid of the same class, where a class is defined by common physicochemical amino acid side chain properties and high substitution frequencies in homologous proteins found in nature, as determined, for example, by a standard Dayhoff frequency exchange matrix or BLOSUM matrix. Six general classes of amino acid side chains have been categorized and include: Class I (Cys); Class II (Ser, Thr, Pro, Ala, Gly); Class III (Asn, Asp, Gln, Glu); Class IV (His, Arg, Lys); Class V (lie, Leu, Val, Met); and Class VI (Phe, Tyr, Trp). For example, substitution of an Asp for another class III residue such as Asn, Gin, or Glu, is a conservative substitution.

The term “Non-conservative substitution”—refers to the substitution of an amino acid in one class with an amino acid from another class; for example, substitution of an Ala, a class II residue, with a class III residue such as Asp, Asn, Glu, or Gin.

By “Chemically modified”—when referring to the product of the invention, is meant a product (polypeptide) where at least one of its amino acid residues is modified either by natural processes, such as processing or other post-translational modifications, or by chemical modification techniques which are well known in the art. Among the numerous known modifications typical, but not exclusive examples include: acetylation, acylation, amidation, ADP-ribosylation, glycosylation, GPI anchor formation, covalent attachment of a lipid or lipid derivative, methylation, myristlyation, pegylation, prenylation, phosphorylation, ubiqutination, or any similar process.

The term “Expression vector”—refers to vectors that have the ability to incorporate and express heterologous DNA fragments in a foreign cell. Many prokaryotic and eukaryotic expression vectors are known and/or commercially available. Selection of appropriate expression vectors is within the knowledge of those having skill in the art.

Throughout this application, various publications, including United States patents, are referenced by author and year and patents by number. Full citations for the publications are listed below. The disclosures of these publications and patents in their entireties are hereby incorporated by reference into this application in order to more fully describe the state of the art to which this invention pertains.

The invention has been described in an illustrative manner, and it is to be understood that the terminology which has been used is intended to be in the nature of words of description rather than of limitation.

Obviously, many modifications and variations of the present invention are possible in light of the above teachings. It is, therefore, to be understood that within the scope of the appended claims, the invention can be practiced otherwise than as specifically described.

### BRIEF DESCRIPTION OF THE FIGURES

FIGS. 1A-1C depicts the nucleic acid sequence of the long splice variant of human STR50 (SEQ ID NO:1).

FIG. 2 depicts the amino acid sequence of the long splice variant of human STR50 (SEQ ID NO:2).

FIGS. 3A-3C depicts the nucleic acid sequence of the short splice variant of human STR50 (SEQ ID NO:3).

FIG. 4 depicts the amino acid sequence of the short splice variant of human STR50 (SEQ ID NO:4).

### EXAMPLES

Without further elaboration, it is believed that one skilled in the art can, using the preceding description, utilize the present invention to its fullest extent. The following preferred specific embodiments are, therefore, to be construed as merely illustrative, and not limitative of the claimed invention in any way.

Standard molecular biology protocols known in the art not specifically described herein are generally followed essentially as in Sambrook et al., Molecular cloning: A laboratory manual, Cold Springs Harbor Laboratory, New-York (1989, 1992), and in Ausubel et al., Current Protocols in Molecular Biology, John Wiley and Sons, Baltimore, Md. (1988).

### Example 1

### Identification and Preparation of Key Genes Involved in the Stroke Event

As a first step to the novel drug discovery, key genes involved in the stroke event were identified, as provided by the following methods:

Summary of cDNA Micro-Array Construction

The polynucleotide encoding STR50 was found by microarray-based differential gene expression, evaluated by both in vivo and in vitro models.
The cDNA microarray was constructed by combining cDNA libraries (Table 1), including a subtraction library, enriched for stroke specific genes. As a result, the “Stroke Chip” consists of a microarray imprinted with about 10,000 low-redundant stroke-specific cDNA clones. The libraries printed on the chip were as described in Table 1.

TABLE 1
Design of the Stroke Chip: Library types and cDNA sources.

| | Material | | Time points | | | |
|---|---|---|---|---|---|---|
| Type of Library | In vivo | In vitro | 3 h | 6 h | 16 h | 24 h |
| Subtraction library | [MCAO] − [Sham] | | +L3 | +L4 | | |
| (five independent | [MCAO + FK506] − | | +L5 | +L6 | | |
| libraries) | [MCAO] | | | | | |
| | | Primary neurons: | +L1 | +L1 | +L1 | +L1 |
| | | [Hypoxia + FK506] − | | | | |
| | | [Normoxia + FK506] | | | | |
| SDGI library | MCAO | | +L7 | +L8 | | |
| (pod of 6 | MCAO + FK506 | | +L9 | | | |
| conditions) | Sham + FK506 | | +L10 | | | |
| | | Primary neurons: | +L2 | +L2 | +L2 | +L2 |
| | | [Hypoxia] | | | | |
| | | Primary neurons: | +L11 | | | |
| | | [Hypoxia + FK506] | | | | |

Each library is indicated by L and numbered. Middle cerebral artery occlusion (MCAO) was performed in SD rats and primary neurons are rat cortical primary neurons. Normoxia indicates normal oxygen concentration.

FK506 (tacrolimus) is a known immunosuppressive agent produced by Streptomyces tsukubaesis. FK506 possesses neuroprotective activity by delaying or preventing hypoxia-induced death of neuronal cells. In addition, it can cause re-growth of damaged nerve cells. The specific molecular mechanism underlying the neuroprotective activity of FK506 is largely unknown although there are indications for suppression of activities of calcineurin and nitric oxide synthase as well as prevention of stroke induced generation of ceramide and Fas signaling. In the present invention, FK 506 serves for pinpointing genes that are not only regulated by ischemic-induced damage but are also regulated by the addition of FK-506.

The libraries imprinted on the Stroke Chip were constructed as follows:

a) Subtractive libraries: An ischemia (stroke) model was created in SD and SHR rats by permanent middle cerebral artery occlusion (MCAO). Control rats of the same strain were subjected to a sham operation (Sham). Half of the rats of each group were given FK506 treatment at 0 hour. Subtraction libraries comprised genes expressed in the MCAO rats but not in the sham operated rats (MCAO—Sham), and those genes expressed in the MCAO rats treated with FK506 (taken at 3 hours and 6 hours after FK506 treatment) but not in the MCAO treated rats ([MCAO+FK506]−[MCAO]). Another library included in the Stroke Chip was derived from in vitro treatment of primary neurons from the cerebellum of 7-day rat pups. The cells were subjected to hypoxia (0.5% O₂) for 16 hours. The cells under hypoxia and control cells under normal oxygen concentration (normoxia) were treated with FK506 (100 ng/ml) at 0 hour and the cDNA extracted after 16 hours. A subtraction library was made from the cDNA fragments expressed in the FK506 treated cells under hypoxia but not in the FK506 treated cells under normoxia ([Hypoxia+FK506]-[Normoxia+FK506]).
b) Libraries generated by sequence-dependent gene identification (SDGI). This technique is essentially as described in PCT application no. PCT/US01/09392. SDGI libraries were prepared from brain tissues of the rats subjected to MCAO, MCAO rats three and six hours after treatment with FK506, and sham operated rats three and six hours after treatment with FK506. SDGI libraries were also prepared from primary neurons that were subjected to hypoxia for 16 hours in the in vitro experiments and from primary neurons, pretreated with FK506 and subjected to hypoxia for 16 hours.

Thus, the cDNA libraries used in the preparation of the stroke chip were prepared as described above, and so were enriched for cDNAs that are differentially expressed in stroke by either subtractive hybridization (SSH) and/or sequence-dependent gene identification (SDGI).

The stroke chip was used for differential hybridization experiments as described below.

### Hybridizations to the Stroke Chip

Cells either in vivo or in vitro were subjected to a developmental, physiological, pharmacological or other cued event that would cause genes to be activated or repressed in response thereto (this gene expression array technology was disclosed, for example in U.S. Pat. No. 5,807,522), and probes were produced; production of probes and their use in interrogating a microarray chip is described for example in U.S. Pat. No. 6,291,170.

Hybridizations were performed according to the following:

Probes used for hybridizations on the Stroke chip were prepared using Four paired groups of animals treated by the following treatments:
1. Animals that in addition to MCAO received FK-506 were sacrificed at 1.5, 3 and 6 hours. The cortex of these animals was removed and used for probe generation
2. Animals that in addition to MCAO received FK-506 were sacrificed at 1.5, 3 and 6 hours. The whole ipsilateral hemisphere (the operated side) of these animals was removed and used for probe generation
3. Animals that in addition to MCAO received vehicle were sacrificed at 1.5, 3, 6, 12, 24 and 48 hours. The ipsilateral cortex of these animals was removed and used for probe generation
4. Animals that in addition to MCAO received FK-506 were sacrificed at 1.5, 3, 6, 12, 24 and 48 hours. The whole ipsilateral hemisphere of these animals was removed and used for probe generation.

The probes were labeled and hybridized to the stroke chip.

In addition to these probes, a common control probe labeled with Cy3 was added to each hybridization. The common control probes were mixtures of poly-A RNA extracted from the whole brain of SD rats.

Preparation of Tissues for In Situ Analysis Coronal sections were prepared from paraffin blocks of sham operated rat brains and brains subjected to MCAO.

The model was characterized using hybridization of control genes known to be affected in stroke such as c-fos and p21 and staining of sections with microtubule associated protein 2 (stains neuronal cell body and dendrites indicating the integrity of neuronal cell cytoskeleton) GFAP (glial filament associated protein); this staining is specific for astrocytes and not myelinating oligodendrocytes and indicates the integrity of glial cell cytoskeleton. Results of these hybridizations were consistent with previously reported results. Thus, suitability of obtained paraffin blocks for in situ hybridization study and suitability of the model for this study were demonstrated.

### Summary of the Results

CRP binds to a range of substances such as phosphorylcholine, fibronectin, chromatin, histones, and ribonucleoprotein in a calcium-dependent manner. It is a ligand for specific receptors on phagocytic leukocytes, mediates activation reactions on monocytes and macrophages, and activates its complement. Plasma CRP is the classical acute-phase protein, increasing 1,000-fold in response to infection, ischemia, trauma, burns, and inflammatory conditions.

The novel gene STR50 was found to be up-regulated at 12 hours (2.1 folds in cortex, 2.5 folds in whole hemisphere), 24 hours (3.3 fold in cortex and 3 folds in whole hemisphere) and 48 hours (1.9 folds in cortex and 2.6 folds in whole hemisphere); FK-506 had no effect in the regulation of this gene, in MCAO or in sham-operated animals (1.5, 3, 6, and 48 hours).

### In Situ Experiments Performed in Coronal Sections of MCAO

The ³⁵S-labeled probe specific to the STR50 gene was hybridized to coronal section of rat brains fixed at different time points (1.5 hr, 3 hr, 6 hr, 12 hr, 24 hr, 48 hr, 72 hr and 96 hr) after permanent middle cerebral artery occlusion (MCAO) or sham operation. Results of this in situ hybridization study show little or no expression in normal brain. STR50 shows weak neuronal activation in the frontal cortex at 1.5 hr; at 6-24 hr the neuronal expression spreads throughout the periinfarct area in parallel with the intracellular relocation of the mRNA; By 48-72 hr, neuronal expression is Down-regulated.

In addition, non-neuronal expression was observed in vascular tissue (at 6-96 hr) and infiltrating cells (24-96 hr). Thus, the results of the DNA microarray based experiments were confirmed by the in situ hybridization studies.

### Example 2

### General Methods

### General Methods in Molecular Biology

Standard molecular biology techniques known in the art and not specifically described were generally followed as in Sambrook et al., Molecular Cloning: A Laboratory Manual, Cold Springs Harbor Laboratory, New York (1989, 1992), and in Ausubel et al., Current Protocols in Molecular Biology, John Wiley and Sons, Baltimore, Md. (1989).

Polymerase chain reaction (PCR) was carried out generally as in PCR Protocols: A Guide To Methods And Applications, Academic Press, San Diego, Calif. (1990). Reactions and manipulations involving other nucleic acid techniques, unless stated otherwise, were performed as generally described in Sambrook et al., 1989, Molecular Cloning: A Laboratory Manual, Cold Spring Harbor Laboratory Press, and methodology as set forth in U.S. Pat. Nos. 4,666,828; 4,683,202; 4,801,531; 5,192,659 and 5,272,057 and incorporated herein by reference.

Protein Purification is performed as described below in Example 3.

Vectors are constructed containing the cDNA of the present invention by those skilled in the art and can contain all expression elements necessary to achieve the desired transcription of the sequences, should transcription be required (see below in specific methods for a more detailed description). Other beneficial characteristics can also be contained within the vectors such as mechanisms for recovery of the nucleic acids in a different form. Phagemids are a specific example of such beneficial vectors because they can be used either as plasmids or as bacteriophage vectors. Examples of other vectors include viruses such as bacteriophages, baculoviruses and retroviruses, DNA viruses, cosmids, plasmids, liposomes and other recombination vectors. The vectors can also contain elements for use in either procaryotic or eucaryotic host systems. One of ordinary skill in the art knows which host systems are compatible with a particular vector. The vectors are introduced into cells or tissues by any one of a variety of known methods within the art (calcium phosphate transfection; electroporation; lipofection; protoplast fusion; polybrene transfection). The host cell can be any eucaryotic and procaryotic cells, which can be transformed with the vector and which supports the production of the polypeptide. Methods for transformation can be found generally described in Sambrook et al., Molecular Cloning: A Laboratory Manual, Cold Springs Harbor Laboratory, New York (1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor, Mich. (1995) and Gilboa, et al. (1986) and include, for example, stable or transient transfection, lipofection, electroporation and infection with recombinant viral vectors. In addition, see U.S. Pat. No. 4,866,042 for vectors involving the central nervous system and also U.S. Pat. Nos. 5,464,764 and 5,487,992 for positive-negative selection methods.

### General Methods in Immunology

Standard methods in immunology known in the art and not specifically described were generally followed as in Stites et al.(eds), Basic and Clinical Immunology (8th Edition), Appleton & Lange, Norwalk, Conn. (1994) and Mishell and Shiigi (eds), Selected Methods in Cellular Immunology, W.H. Freeman and Co., New York (1980).

### Immunoassays

In general, ELISAs are the preferred immunoassays employed to assess a specimen. ELISA assays are well known to those skilled in the art. Both polyclonal and monoclonal antibodies can be used in the assays. Where appropriate other immunoassays, such as radioimmunoassays (RIA) can be used as are known to those in the art. Available immunoassays are extensively described in the patent and scientific literature. See, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521 as well as Sambrook et al, Molecular Cloning: A Laboratory Manual, Cold Springs Harbor, N.Y., 1989.

### Example 3

### Preparation of Polypeptides

Polypeptides may be produced via several methods, for example:

### 1) Synthetically;

Synthetic polypeptides can be made using a commercially available machine, using the known sequence of the desired polypeptide.

### 2) Recombinant Methods:

A preferred method of making polypeptides is to clone a polynucleotide comprising the cDNA of the gene of the desired polypeptide into an expression vector and culture the cell harboring the vector so as to express the encoded polypeptide, and then purify the resulting polypeptide, all performed using methods known in the art as described in, for example, Marshak et al., “Strategies for Protein Purification and Characterization. A laboratory course manual.” CSHL Press (1996). (in addition, see Bibl Haematol. 1965; 23:1165-74 Appl Microbiol. 1967 July; 15(4):851-6; Can J. Biochem. 1968 May; 46(5):441-4; Biochemistry. 1968 July; 7(7):2574-80; Arch Biochem Biophys. 1968 Sep. 10; 126(3):746-72; Biochem Biophys Res Commun. 1970 Feb. 20; 38(4):825-30).).

The expression vector can include a promoter for controlling transcription of the heterologous material and can be either a constitutive or inducible promoter to allow selective transcription. Enhancers that can be required to obtain necessary transcription levels can optionally be included. The expression vehicle can also include a selection gene.

Vectors can be introduced into cells or tissues by any one of a variety of methods known within the art. Such methods can be found generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Vega et al., Gene Targeting, CRC Press, Ann Arbor, Mich. (1995), Vectors: A Survey of Molecular Cloning Vectors and Their Uses, Butterworths, Boston Mass. (1988) and Gilboa et al. (1986).

3) Purification from Natural Sources:

Desired polypeptides can be purified from natural sources (such as tissues) using many methods known to one of ordinary skill in the art, such as for example: immuno-precipitation, or matrix-bound affinity chromatography with any molecule known to bind the desired polypeptide.

Protein purification is practiced as is known in the art as described in, for example, Marshak et al., “Strategies for Protein Purification and Characterization. A laboratory course manual.” CSHL Press (1996).

The claimed polypeptides can be used for diagnosis and treatment as described above. Additionally, the claimed polypeptides can be used for the manufacture of antibodies as described below in Example 5; said antibodies can be used to diagnose cells which have undergone neurotoxic stress and, as a result, express an abnormal amount of the STR50 polypeptide as compared with normal cells. Furthermore, it is well-known that proteins and polypeptides are nutritious and edible.

### Example 4

### Preparation of Polynucleotides

The polynucleotides of the subject invention can be constructed by using a commercially available DNA synthesizing machine; overlapping pairs of chemically synthesized fragments of the desired gene can be ligated using methods well known in the art (e.g., see U.S. Pat. No. 6,121,426).

Another means of isolating a polynucleotide is to obtain a natural or artificially designed DNA fragment based on that sequence. This DNA fragment is labeled by means of suitable labeling systems which are well known to those of skill in the art; see, e.g., Davis et al. (1986). The fragment is then used as a probe to screen a lambda phage cDNA library or a plasmid cDNA library using methods well known in the art; see, generally, Sambrook et al., Molecular Cloning: A Laboratory Manual, Cold Spring Harbor Laboratory Press, New York (1989), in Ausubel et al., Current Protocols in Molecular Biology, John Wiley and Sons, Baltimore, Md. (1989),

Colonies can be identified which contain clones related to the cDNA probe and these clones can be purified by known methods. The ends of the newly purified clones are then sequenced to identify full-length sequences. Complete sequencing of full-length clones is performed by enzymatic digestion or primer walking. A similar screening and clone selection approach can be applied to clones from a genomic DNA library.

### Example 5

### Preparation of Anti-STR50 Antibodies

Antibodies which bind to STR50 may be prepared using an intact polypeptide or fragments containing smaller polypeptides as the immunizing antigen. For example, it may be desirable to produce antibodies that specifically bind to the N- or C-terminal or any other suitable domains of the STR50. The polypeptide used to immunize an animal can be derived from translated cDNA or chemical synthesis which can be conjugated to a carrier protein, if desired. Such commonly used carriers which are chemically coupled to the polypeptide include keyhole limpet hemocyanin (KLH), thyroglobulin, bovine serum albumin (BSA) and tetanus toxoid. The coupled polypeptide is then used to immunize the animal.

If desired, polyclonal or monoclonal antibodies can be further purified, for example by binding to and elution from a matrix to which the polypeptide or a peptide to which the antibodies were raised is bound. Those skilled in the art know various techniques common in immunology for purification and/or concentration of polyclonal as well as monoclonal antibodies (Coligan et al, Unit 9, Current Protocols in Immunology, Wiley Interscience, 1994).

Methods for making antibodies of all types, including fragments, are known in the art (See for example, Harlow and Lane, Antibodies: A Laboratory Manual, Cold Spring Harbor Laboratory, New York (1988)). Methods of immunization, including all necessary steps of preparing the immunogen in a suitable adjuvant, determining antibody binding, isolation of antibodies, methods for obtaining monoclonal antibodies, and humanization of monoclonal antibodies are all known to the skilled artisan The antibodies may be humanized antibodies or human antibodies. Antibodies can be humanized using a variety of techniques known in the art including CDR-grafting (EP239,400: PCT publication WO.91/09967; U.S. Pat. Nos. 5,225,539; 5,530,101; and 5,585,089, veneering or resurfacing (EP 592,106; EP 519,596; Padlan, Molecular Immunology 28(4/5):489-498 (1991); Studnicka et al., Protein Engineering 7(6):805-814 (1994); Roguska et al., PNAS 91:969-973 (1994)), and chain shuffling (U.S. Pat. No. 5,565,332).

The monoclonal antibodies as defined include antibodies derived from one species (such as murine, rabbit, goat, rat, human, etc.) as well as antibodies derived from two (or more) species, such as chimeric and humanized antibodies.

Completely human antibodies are particularly desirable for therapeutic treatment of human patients. Human antibodies can be made by a variety of methods known in the art including phage display methods using antibody libraries derived from human immunoglobulin sequences. See also U.S. Pat. Nos. 4,444,887 and 4,716,111; and PCT publications WO 98/46645, WO 98/50433, WO 98/24893, WO 98/16654, WO 96/34096, WO 96/33735, and WO 91/10741; each of which is incorporated herein by reference in its entirety.

Additional information regarding all types of antibodies, including humanized antibodies, human antibodies and antibody fragments can be found in WO 01/05998, which is incorporated herein by reference in its entirety.

### Example 6

### Cloning Process of the STR50 Gene

The Initial sequence isolated from the stroke chip screen as described at length in Example 1 was a 517 bp long rat fragment. This sequence was used as a query in a search against a mouse EST database, and was found to be similar to a few ESTs.

The extended consensus sequence, which was created from the alignment of the query sequence with the similar EST sequences, served as a query in repeated searches that yielded more partially overlapping ESTs.

The final result of the repeated searches was a contig of mouse sequences.

This contig was then physically verified by the RT-PCR method, using mRNA templates derived from both mouse and rat tissues.

The mouse consensus sequence achieved through RT-PCR was used as the query in a bioinformatic analysis against a human nucleotide sequences database, and was found to be similar to human gi 14248494. The predicted human cDNA in this gi is 3766 bp long with a 2202 bp ORF and the inventors came to the conclusion that it is truncated at the N-terminus.

Therefore, another search was performed, this time against the Human Genome database, and a putative exon that is 5′ to the truncated mouse consensus sequence was found, which extended the sequence and predicted ORF of the gene. No match for this 5′ exon sequence was found in the human EST databases.

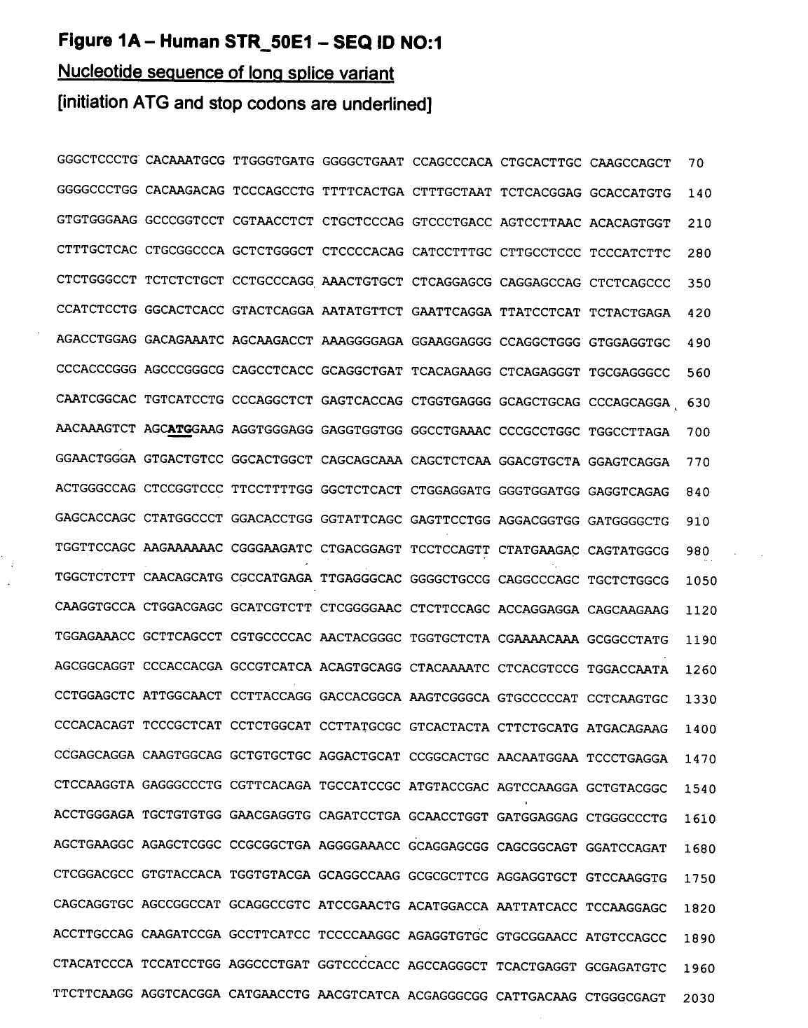

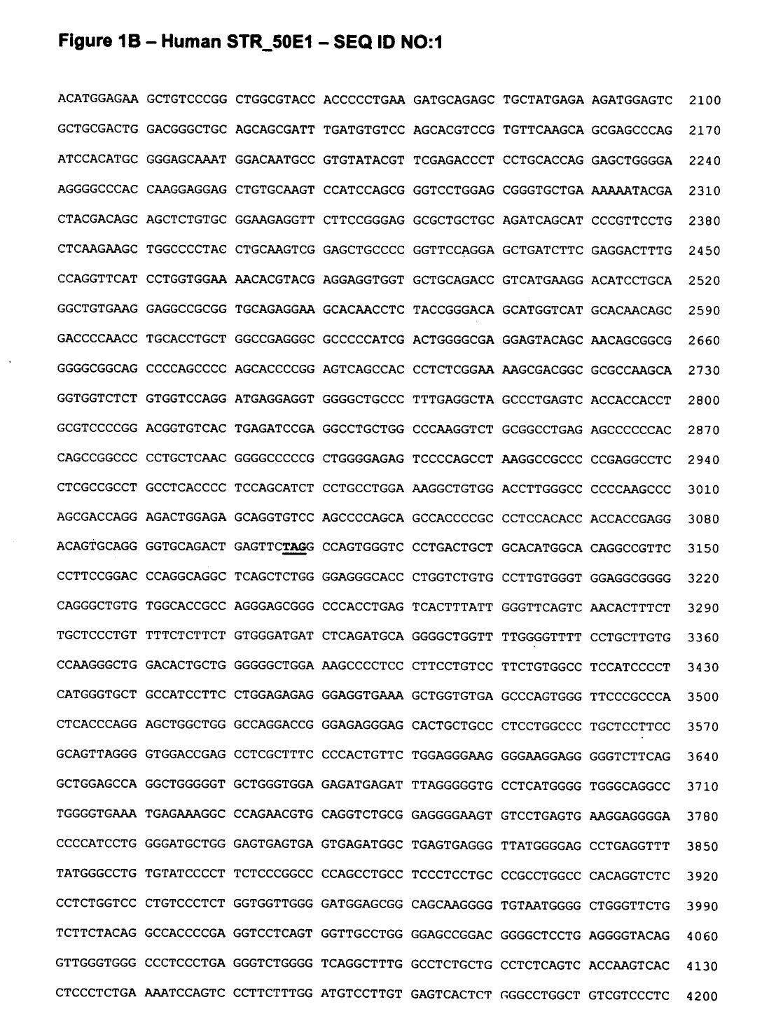

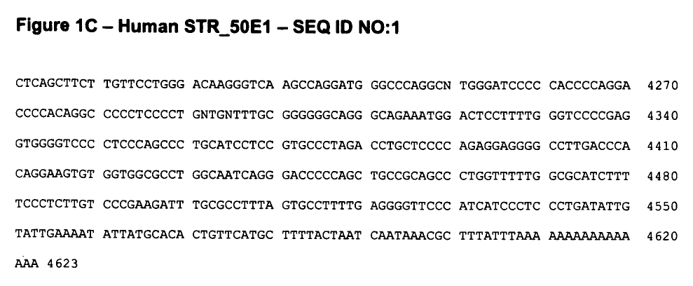

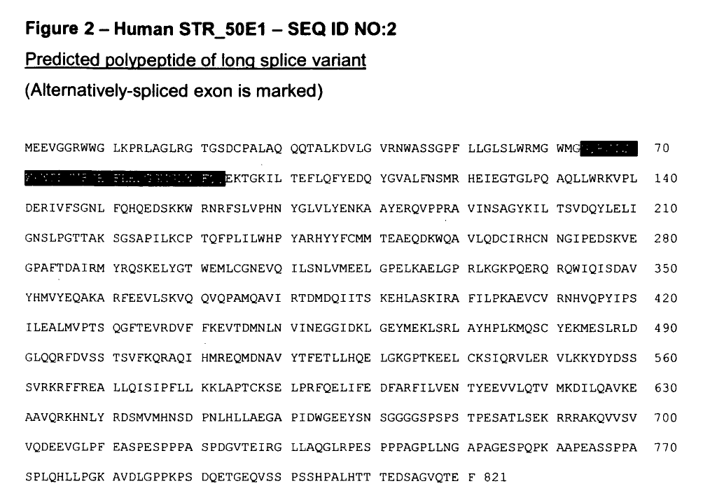

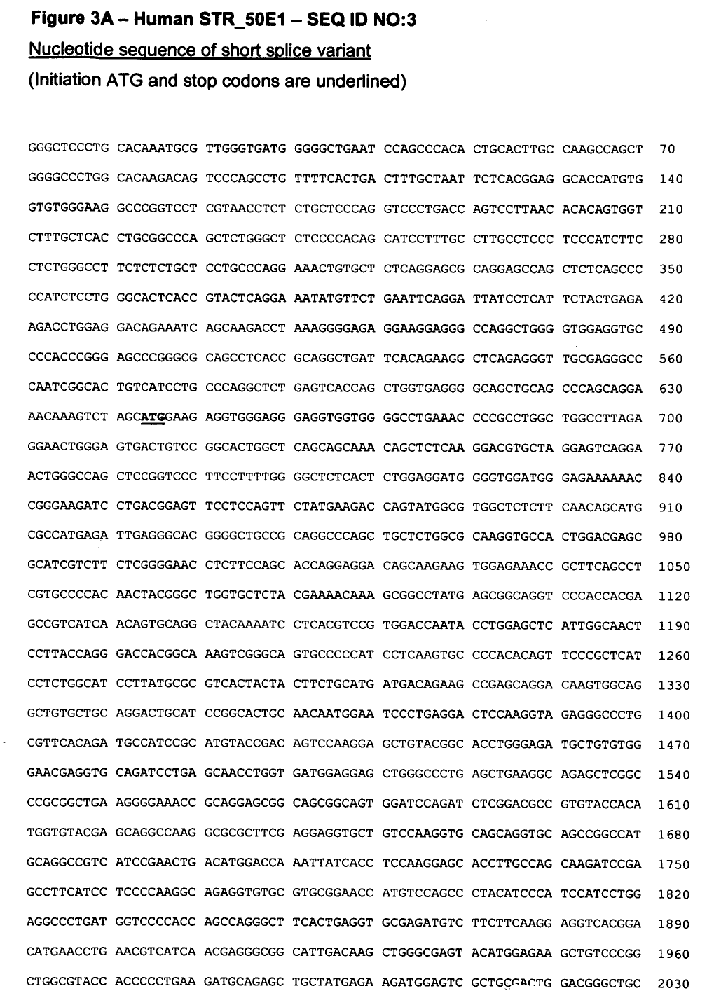

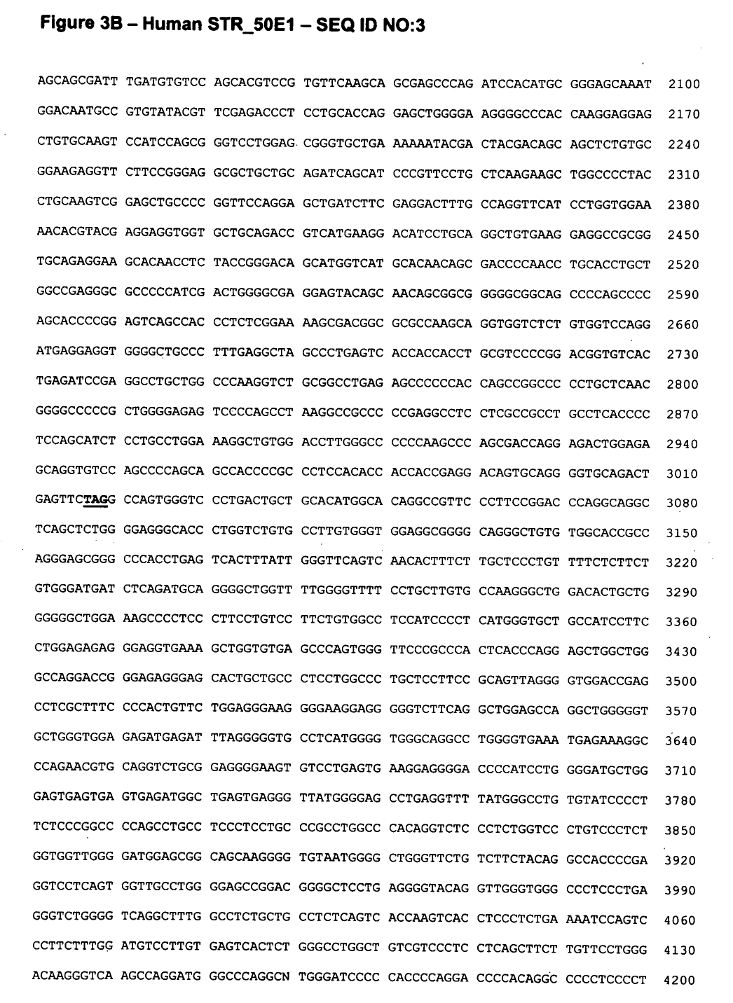

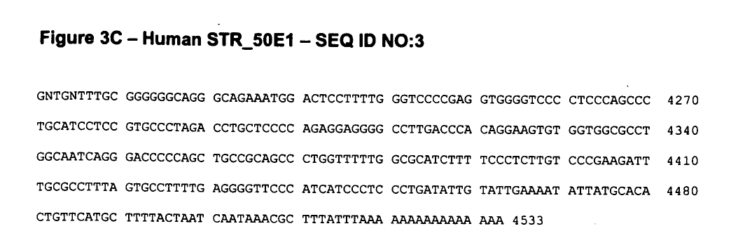

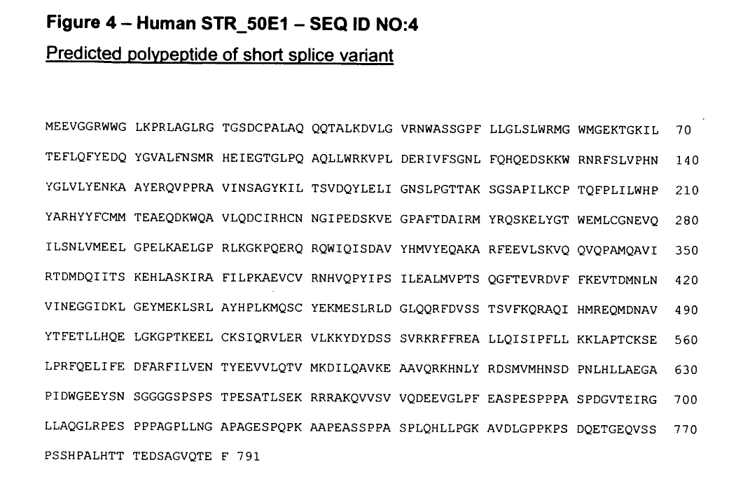

The putative extension of the human gene sequence was verified by performing an RT-PCR reaction on human mRNA, which yielded two products, representing two splice variants (FIGS. 1 and 3):

FIGS. 1A-1C represents the long variant, which contains an additional exon; this variant is 4008 bp long, with a 2463 bp long ORF which translates into a 821 amino acid long polypeptide.

FIGS. 3A-3C represents the short variant which is 4533 bp long with 2373 bp ORF which translates into a 791 amino acid long polypeptide.

### Example 7

### Screening Systems

The STR50 gene or polypeptide may be used in a screening assay for identifying and isolating compounds which modulate its activity and, in particular, compounds which modulate neurotoxic stress or neurodegenerative diseases. The compounds to be screened comprise inter alia substances such as small chemical molecules, antibodies, antisense oligonucleotides, antisense DNA or RNA molecules, polypeptides and dominant negatives, and expression vectors.

Many types of screening assays are known to those of ordinary skill in the art. The specific assay which is chosen depends to a great extent on the activity of the candidate gene or the polypeptide expressed thereby. Thus, if it is known that the expression product of a candidate gene has enzymatic activity, then an assay which is based on inhibition (or stimulation) of the enzymatic activity can be used. If the candidate polypeptide is known to bind to a ligand or other interactor, then the assay can be based on the inhibition of such binding or interaction. When the candidate gene is a known gene, then many of its properties can also be known, and these can be used to determine the best screening assay. If the candidate gene is novel, then some analysis and/or experimentation is appropriate in order to determine the best assay to be used to find inhibitors of the activity of that candidate gene. The analysis can involve a sequence analysis to find domains in the sequence which shed light on its activity.

As is well known in the art, the screening assays can be cell-based or non-cell-based. The cell-based assay is performed using eukaryotic cells such as HeLa cells, and such cell-based systems are particularly relevant in order to directly measure the activity of candidate genes which are anti-apoptotic functional genes, i.e., expression of the gene prevents apoptosis or otherwise prevents cell death in target cells. One way of running such a cell-based assay uses tetracycline-inducible (Tet-inducible) gene expression. Tet-inducible gene expression is well known in the art; see for example, Hofmann et al, 1996, Proc Natl Acad Sci 93(11):5185-5190.

Tet-inducible retroviruses have been designed incorporating the Self-inactivating (SIN) feature of a 3′ Ltr enhancer/promoter retroviral deletion mutant. Expression of this vector in cells is virtually undetectable in the presence of tetracycline or other active analogs. However, in the absence of Tet, expression is turned on to maximum within 48 hours after induction, with uniform increased expression of the whole population of cells that harbor the inducible retrovirus, thus indicating that expression is regulated uniformly within the infected cell population.

If the gene product of the candidate gene phosphorylates with a specific target protein, a specific reporter gene construct can be designed such that phosphorylation of this reporter gene product causes its activation, which can be followed by a color reaction. The candidate gene can be specifically induced, using the Tet-inducible system discussed above, and a comparison of induced versus non-induced genes provides a measure of reporter gene activation.

In a similar indirect assay, a reporter system can be designed that responds to changes in protein-protein interaction of the candidate protein. If the reporter responds to actual interaction with the candidate protein, a color reaction occurs.

One can also measure inhibition or stimulation of reporter gene activity by modulation of its expression levels via the specific candidate promoter or other regulatory elements. A specific promoter or regulatory element controlling the activity of a candidate gene is defined by methods well known in the art. A reporter gene is constructed which is controlled by the specific candidate gene promoter or regulatory elements. The DNA containing the specific promoter or regulatory agent is actually linked to the gene encoding the reporter. Reporter activity depends on specific activation of the promoter or regulatory element. Thus, inhibition or stimulation of the reporter is a direct assay of stimulation/inhibition of the reporter gene; see, for example, Komarov et al (1999), Science vol 285, 1733-7 and Storz et al (1999) Analytical Biochemistry, 276, 97-104.

Various non-cell-based screening assays are also well within the skill of those of ordinary skill in the art. For example, if enzymatic activity is to be measured, such as if the candidate protein has a kinase activity, the target protein can be defined and specific phosphorylation of the target can be followed. The assay can involve either inhibition of target phosphorylation or stimulation of target phosphorylation, both types of assay being well known in the art; for example see Mohney et al (1998) J. Neuroscience 18, 5285 and Tang et al (1997) J Clin. Invest. 100, 1180 for measurement of kinase activity. Although this is not relevant in the case of STR50 which does not have an enzymatic activity, there is a possibility that STR50 interacts with an enzyme and regulates its enzymatic activity through protein-protein interaction.

One can also measure in vitro interaction of a candidate polypeptide with interactors. In this screen, the candidate polypeptide is immobilized on beads. An interactor, such as a receptor ligand, is radioactively labeled and added. When it binds to the candidate polypeptide on the bead, the amount of radioactivity carried on the beads (due to interaction with the candidate polypeptide) can be measured. The assay indicates inhibition of the interaction by measuring the amount of radioactivity on the bead.

Any of the screening assays, according to the present invention, can include a step of identifying the chemical compound (as described above) or other species which tests positive in the assay and can also include the further step of producing as a medicament that which has been so identified. It is considered that medicaments comprising such compounds, or chemical analogs or homologs thereof, are part of the present invention. The use of any such compounds identified for inhibition or stimulation of apoptosis is also considered to be part of the present invention.

### Example 8

### Pharmacology and Drug Delivery

The species or compound of the present invention is administered and dosed in accordance with good medical practice, taking into account the clinical condition of the individual patient, the disease to be treated, the site and method of administration, scheduling of administration, patient age, sex, body weight and other factors known to medical practitioners. The pharmaceutically “effective amount” for purposes herein is thus determined by such considerations as are known in the art. The amount must be effective to achieve improvement including but not limited to improved survival rate or more rapid recovery, or improvement or elimination of symptoms and other indicators as are selected as appropriate measures by those skilled in the art. The treatment generally has a length proportional to the length of the disease process and drug effectiveness and the patient species being treated. It is noted that humans are treated generally longer than the mice or other experimental animals exemplified herein.

The species or compound of the present invention can be administered by any of the conventional routes of administration. It should be noted that it can be administered as the compound or as pharmaceutically acceptable salt and can be administered alone or as an active ingredient in combination with pharmaceutically acceptable carriers, solvents, diluents, excipients, adjuvants and vehicles. The compounds can be administered orally, subcutaneously or parenterally including intravenous, intraarterial, intramuscular, intraperitoneally, and intranasal administration as well as intrathecal and infusion techniques. Implants of the compounds are also useful. Liquid forms may be prepared for injection, the term including subcutaneous, transdermal, intravenous, intramuscular, intrathecal, and other parental routes of administration. The liquid compositions include aqueous solutions, with and without organic cosolvents, aqueous or oil suspensions, emulsions with edible oils, as well as similar pharmaceutical vehicles. In addition, under certain circumstances the compositions for use in the novel treatments of the present invention may be formed as aerosols, for intranasal and like administration. The patient being treated is a warm-blooded animal and, in particular, mammals including man. The pharmaceutically acceptable carriers, solvents, diluents, excipients, adjuvants and vehicles as well as implant carriers generally refer to inert, non-toxic solid or liquid fillers, diluents or encapsulating material not reacting with the active ingredients of the invention.

When administering the compound of the present invention parenterally, it is generally formulated in a unit dosage injectable form (solution, suspension, emulsion). The pharmaceutical formulations suitable for injection include sterile aqueous solutions or dispersions and sterile powders for reconstitution into sterile injectable solutions or dispersions. The carrier can be a solvent or dispersing medium containing, for example, water, ethanol, polyol (for example, glycerol, propylene glycol, liquid polyethylene glycol, and the like), suitable mixtures thereof, and vegetable oils. Proper fluidity can be maintained, for example, by the use of a coating such as lecithin, by the maintenance of the required particle size in the case of dispersion and by the use of surfactants. Nonaqueous vehicles such a cottonseed oil, sesame oil, olive oil, soybean oil, corn oil, sunflower oil, or peanut oil and esters, such as isopropyl myristate, can also be used as solvent systems for compound compositions. Additionally, various additives which enhance the stability, sterility, and isotonicity of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. In many cases, it is desirable to include isotonic agents, for example, sugars, sodium chloride, and the like. Prolonged absorption of the injectable pharmaceutical form can be brought about by the use of agents delaying absorption, for example, aluminum monostearate and gelatin. According to the present invention, however, any vehicle, diluent, or additive used have to be compatible with the compounds.

Sterile injectable solutions can be prepared by incorporating the compounds utilized in practicing the present invention in the required amount of the appropriate solvent with various of the other ingredients, as desired.

A pharmacological formulation of the present invention can be administered to the patient in an injectable formulation containing any compatible carrier, such as various vehicle, adjuvants, additives, and diluents; or the compounds utilized in the present invention can be administered parenterally to the patient in the form of slow-release subcutaneous implants or targeted delivery systems such as monoclonal antibodies, vectored delivery, iontophoretic, polymer matrices, liposomes, and microspheres. Examples of delivery systems useful in the present invention include U.S. Pat. Nos. 5,225,182; 5,169,383; 5,167,616; 4,959,217; 4,925,678; 4,487,603; 4,486,194; 4,447,233; 4,447,224; 4,439,196; and 4,475,196. Many other such implants, delivery systems, and modules are well known to those skilled in the art.

A pharmacological formulation of the compound utilized in the present invention can be administered orally to the patient. Conventional methods such as administering the compounds in tablets, suspensions, solutions, emulsions, capsules, powders, syrups and the like are usable. Known techniques which deliver it orally or intravenously and retain the biological activity are preferred. In one embodiment, the compound of the present invention can be administered initially by intravenous injection to bring blood levels to a suitable level. The patient's levels are then maintained by an oral dosage form, although other forms of administration, dependent upon the patient's condition and as indicated above, can be used.

In general, the active dose of compounds for humans is in the range of from 1 ng/kg to about 20-100 mg/kg body weight per day, preferably about 0.01 mg to about 2-10 mg/kg body weight per day, in a regimen of one dose per day or twice or three or more times per day for a period of 1-2 weeks or longer, preferably for 24-to 48 hrs or by continuous infusion during a period of 1-2 weeks or longer.

It will be appreciated that the most appropriate administration of the pharmaceutical compositions of the present invention will depend on the type of injury or disease being treated. Thus, the treatment of an acute event will necessitate systemic administration of the active composition comparatively rapidly after induction of the injury. On the other hand, diminution of chronic degenerative damage may necessitate a sustained dosage regimen.

### Example 9

### Experimental Models

CNS injury—The potential of the use of an STR50 modulator for treating CNS injury is evaluated in animal models. The models represent varying levels of complexity, by comparison of control animals to the inhibitor treated animals. The efficacy of such treatment is evaluated in terms of clinical outcome, neurological deficit, dose-response and therapeutic window. Test animals are treated with an STR50 modulator intravenously or subcutanously or per os. Control animals are treated with buffer or pharmaceutical vehicle only. Models used are as follows:
1. Closed Head Injury (CHI)—Experimental TBI produces a series of events contributing to neurological and neurometabolic cascades, which are related to the degree and extent of behavioral deficits. CHI is induced under anesthesia, while a weight is allowed to free-fall from a prefixed height (Chen et al, J. Neurotrauma 13, 557, 1996) over the exposed skull covering the left hemisphere in the midcoronal plane.
2. Transient middle cerebral artery occlusion (MCAO)—a 90 to 120 minutes transient focal ischemia is performed in adult, male Sprague Dawley rats, 300-370 gr. The method employed is the intraluminal suture MCAO (Longa et al., Stroke, 30, 84, 1989, and Dogan et al., J. Neurochem. 72, 765, 1999). Briefly, under halothane anesthesia, a 3-O-nylon suture material coated with Poly-L-Lysine is inserted into the right internal carotid artery (ICA) through a hole in the external carotid artery. The nylon thread is pushed into the ICA to the right MCA origin (20-23 mm). 90-120 minutes later the thread is pulled off, the animal is closed and allowed to recover.
3. Permanent middle cerebral artery occlusion (MCAO)—occlusion is permanent, unilateral-induced by electrocoagulation of MCA. Both methods lead to focal brain ischemia of the ipsilateral side of the brain cortex leaving the contralateral side intact (control). The left MCA is exposed via a temporal craniectomy, as described for rats by Tamura A.et al., J Cereb Blood Flow Metab. 1981; 1:53-60. The MCA and its lenticulostriatal branch are occluded proximally to the medial border of the olfactory tract with microbipolar coagulation. The wound is sutured, and animals returned to their home cage in a room warmed at 26° C. to 28° C. The temperature of the animals is maintained all the time with an automatic thermostat.

Evaluation Process The efficacy of the STR50 modulator is determined by mortality rate, weight gain, infarct volume, short and long term clinical and neurophysichological and behavioral (including feeding behavior) outcomes in surviving animals. Infarct volumes are assessed histologically (Knight et al., Stroke, 25, 1252, 1994, and Mintorovitch et al., Magn. Reson. Med. 18, 39, 1991). The staircase test (Montoya et al., J. Neurosci. Methods 36, 219, 1991) or the motor disability scale according to Bederson's method (Bederson et al., Stroke, 17, 472, 1986) are employed to evaluate the functional outcome following MCAO. The animals are followed for different time points, the longest one being two months. At each time point (24 h, 1 week, 3, 6, 8 weeks), animals are sacrificed and cardiac perfusion with 4% formaldehyde in PBS is performed. Brains are removed and serial coronal 200 μm sections are prepared for processing and paraffin embedding. The sections are stained with suitable dyes such as TCC. The infarct area is measured in these sections using a computerized image analyzer.

Utilization of the STR50 modulator treatment as exemplified in the above animal models provides new possibilities for treatment of human brain injury, whether acute or chronic.

### Example 10

### Therapeutic Delivery of Antisense Fragments

In the practice of the invention, antisense fragments may be used. The length of an antisense fragment is preferably from about 9 to about 4,000 nucleotides, more preferably from about 20 to about 2,000 nucleotides, most preferably from about 50 to about 500 nucleotides.

In order to be effective, the antisense fragments of the present invention must travel across cell membranes. In general, antisense fragments have the ability to cross cell membranes, apparently by uptake via specific receptors. As the antisense fragments are single-stranded molecules, they are to a degree hydrophobic, which enhances passive diffusion through membranes. Modifications may be introduced to an antisense fragment to improve its ability to cross membranes. For instance, the AS molecule may be linked to a group which includes partially unsaturated aliphatic hydrocarbon chain and one or more polar or charged groups such as carboxylic acid groups, ester groups, and alcohol groups. Alternatively, AS fragments may be linked to peptide structures, which are preferably membranotropic peptides. Such modified AS fragments penetrate membranes more easily, which is critical for their function and may, therefore, significantly enhance their activity. Palmityl-linked oligonucleotides have been described by Gerster et al (1998): Quantitative analysis of modified antisense oligonucleotides in biological fluids using cationic nanoparticles for solid-phase extraction. Anal Biochem. 1998 Sep. 10; 262(2):177-84 Geraniol-linked oligonucleotides have been described by Shoji et al (1998): Enhancement of anti-herpetic activity of antisense phosphorothioate oligonucleotides 5′ end modified with geraniol. J Drug Target. 1998; 5(4):261-73. Oligonucleotides linked to peptides, e.g., membranotropic peptides, and their preparation have been described by Soukchareun et al (1998): Use of Nalpha-Fmoc-cysteine(S-thiobutyl) derivatized oligodeoxynucleotides for the preparation of oligodeoxynucleotide-peptide hybrid molecules. Bioconjug Chem. 1998 July-August; 9(4):466-75. Modifications of antisense molecules or other drugs that target the molecule to certain cells and enhance uptake of the oligonucleotide by said cells are described by Wang (1998).

The antisense oligonucleotides of the invention are generally provided in the form of pharmaceutical compositions. These compositions are for use by injection, topical administration, or oral uptake.

The mechanism of action of antisense RNA and the current state of the art on use of antisense tools is reviewed in Kumar et al (1998): Antisense RNA: function and fate of duplex RNA in cells of higher eukaryotes. Microbiol Mol Biol Rev. 1998 December; 62(4):1415-34. There are reviews on the chemical (Crooke, 1995: Progress in antisense therapeutics. Hematol Pathol. 1995; 9(2):59-72.; Uhlmann et al, 1990), cellular (Wagner, 1994: Gene inhibition using antisense oligodeoxynucleotides. Nature. 1994 Nov. 24; 372(6504):333-5.) and therapeutic (Hanania, et al, 1995: Recent advances in the application of gene therapy to human disease. Am J. Med. 1995 November; 99(5):537-52.; Scanlon, et al, 1995: Oligonucleotide-mediated modulation of mammalian gene expression. FASEB J. 1995 October; 9(13):1288-96.; Gewirtz, 1993: Oligodeoxynucleotide-based therapeutics for human leukemias. Stem Cells. 1993 October; 11 Suppl 3:96-103) aspects of this rapidly developing technology. The use of antisense oligonucleotides in inhibition of BMP receptor synthesis has been described by Yeh et al (1998): Inhibition of BMP receptor synthesis by antisense oligonucleotides attenuates OP-1 action in primary cultures of fetal rat calvaria cells. J Bone Miner Res. 1998 December; 13(12):1870-9. The use of antisense oligonucleotides for inhibiting the synthesis of the voltage-dependent potassium channel gene Kv1.4 has been described by Meiri et al (1998) Memory and long-term potentiation (LTP) dissociated: normal spatial memory despite CA1 LTP elimination with Kv1.4 antisense. Proc Natl Acad Sci USA. 1998 Dec. 8; 95(25):15037-42. The use of antisense oligonucleotides for inhibition of the synthesis of Bcl-x has been described by Kondo et al (1998): Antisense telomerase treatment: induction of two distinct pathways, apoptosis and differentiation. FASEB J. 1998 July; 12(10):801-11. The therapeutic use of antisense drugs is discussed by Stix (1998): Shutting down a gene. Antisense drug wins approval. Sci Am. 1998 November; 279(5):46, 50; Flanagan (1998) Antisense comes of age. Cancer Metastasis Rev. 1998 June; 17(2):169-76; Guinot et al (1998) Antisense oligonucleotides: a new therapeutic approach Pathol Biol (Paris). 1998 May; 46(5):347-54, and references therein. Within a relatively short time, ample information has accumulated about the in vitro use of AS nucleotide sequences in cultured primary cells and cell lines as well as for in vivo administration of such nucleotide sequences for suppressing specific processes and changing body functions in a transient manner. Further, enough experience is now available from in vitro and in vivo animal models and from human clinical trials to predict human efficacy.

We claim:

1. A purified polynucleotide which encodes STR50.

2. The polynucleotide of claim 1 comprising consecutive nucleotides having a sequence as set forth in SEQ ID NO:1 and homologs or complements thereof.

3. The polynucleotide of claim 1 comprising consecutive nucleotides having a sequence as set forth in SEQ ID NO:3 and homologs or complements thereof.

4. The polynucleotide of claim 1 comprising consecutive nucleotides having a sequence as set forth from position 644 through position 3109 of SEQ ID NO:1 and homologs or complements thereof.

5. The polynucleotide of claim 1 comprising consecutive nucleotides having a sequence as set forth from position 644 through position 3019 of SEQ ID NO:3 and homologs or complements thereof.

6. A purified polynucleotide which encodes a polypeptide having the sequence set forth in SEQ ID NO:2.

7. A purified polynucleotide which encodes a polypeptide having the sequence set forth in SEQ ID NO:4.

8. A vector comprising a polynucleotide according to claim 1.

9. A cell comprising a vector according to claim 8.

10. An antisense polynucleotide complementary to the polynucleotide of claim 1.

11. A polynucleotide having between 10 and 922 consecutive nucleotides from position 1 to position 922 of SEQ ID NO:1.

12. A polynucleotide having between 10 and 766 consecutive nucleotides from position 1 to position 766 of SEQ ID NO:3.

13. Purified STR50.

14. The polypeptide of claim 13 comprising consecutive amino acids the sequence of which extends from position 1 through position 821 of SEQ ID No:2.

15. The polypeptide of claim 13 comprising consecutive amino acids the sequence of which extends from position 1 through position 791 of SEQ ID No:4.

16. A purified polypeptide encoded by the polynucleotide of claim 11.

17. A purified polypeptide encoded by the polynucleotide of claim 12.

18. A purified polypeptide according to claim 13, having the biological activity of modulating neurotoxic stress.

19. A purified polypeptide having at least 70% homology to, and retaining the biological activity of the polypeptide according to claim 18.

20. An antibody that binds to an epitope on the polypeptide of claim 13.

21. An antibody that binds to an epitope on the polypeptide of claim 14.

22. An antibody that binds to an epitope on the polypeptide of claim 15.

23. An antibody that binds to an epitope on the polypeptide of claim 19.


SEQUENCE LISTING (4 sequences)

SEQ ID NO: 1 - DNA, 4623 bp, Homo sapiens
  CDS (644)..(3109)
  misc_feature (4250)..(4250): n is a, c, g, or t
gggctccctgcacaaatgcgttgggtgatgggggctgaatccagcccacactgcacttgccaagccagctggggccctggcacaagacagtcccagcctgttttcactgactttgctaattctcacggaggcaccatgtggtgtgggaaggcccggtcctcgtaacctctctgctcccaggtccctgaccagtccttaacacacagtggtctttgctcacctgcggcccagctctgggctctccccacagcatcctttgccttgcctccctcccatcttcctctgggccttctctctgctcctgcccaggaaactgtgctctcaggagcgcaggagccagctctcagcccccatctcctgggcactcaccgtactcaggaaatatgttctgaattcaggattatcctcattctactgagaagacctggaggacagaaatcagcaagacctaaaggggagaggaaggagggccaggctggggtggaggtgccccacccgggagcccgggcgcagcctcaccgcaggctgattcacagaaggctcagagggttgcgagggcccaatcggcactgtcatcctgcccaggctctgagtcaccagctggtgaggggcagctgcagcccagcaggaaacaaagtctagcatggaagaggtgggagggaggtggtggggcctgaaaccccgcctggctggccttagaggaactgggagtgactgtccggcactggctcagcagcaaacagctctcaaggacgtgctaggagtcaggaactgggccagctccggtcccttccttttggggctctcactctggaggatggggtggatgggaggtcagaggagcaccagcctatggccctggacacctggggtattcagcgagttcctggaggacggtgggatggggctgtggttccagcaagaaaaaaccgggaagatcctgacggagttcctccagttctatgaagaccagtatggcgtggctctcttcaacagcatgcgccatgagattgagggcacggggctgccgcaggcccagctgctctggcgcaaggtgccactggacgagcgcatcgtcttctcggggaacctcttccagcaccaggaggacagcaagaagtggagaaaccgcttcagcctcgtgccccacaactacgggctggtgctctacgaaaacaaagcggcctatgagcggcaggtcccaccacgagccgtcatcaacagtgcaggctacaaaatcctcacgtccgtggaccaatacctggagctcattggcaactccttaccagggaccacggcaaagtcgggcagtgcccccatcctcaagtgccccacacagttcccgctcatcctctggcatccttatgcgcgtcactactacttctgcatgatgacagaagccgagcaggacaagtggcaggctgtgctgcaggactgcatccggcactgcaacaatggaatccctgaggactccaaggtagagggccctgcgttcacagatgccatccgcatgtaccgacagtccaaggagctgtacggcacctgggagatgctgtgtgggaacgaggtgcagatcctgagcaacctggtgatggaggagctgggccctgagctgaaggcagagctcggcccgcggctgaaggggaaaccgcaggagcggcagcggcagtggatccagatctcggacgccgtgtaccacatggtgtacgagcaggccaaggcgcgcttcgaggaggtgctgtccaaggtgcagcaggtgcagccggccatgcaggccgtcatccgaactgacatggaccaaattatcacctccaaggagcaccttgccagcaagatccgagccttcatcctccccaaggcagaggtgtgcgtgcggaaccatgtccagccctacatcccatccatcctggaggccctgatggtccccaccagccagggcttcactgaggtgcgagatgtcttcttcaaggaggtcacggacatgaacctgaacgtcatcaacgagggcggcattgacaagctgggcgagtacatggagaagctgtcccggctggcgtaccaccccctgaagatgcagagctgctatgagaagatggagtcgctgcgactggacgggctgcagcagcgatttgatgtgtccagcacgtccgtgttcaagcagcgagcccagatccacatgcgggagcaaatggacaatgccgtgtatacgttcgagaccctcctgcaccaggagctggggaaggggcccaccaaggaggagctgtgcaagtccatccagcgggtcctggagcgggtgctgaaaaaatacgactacgacagcagctctgtgcggaagaggttcttccgggaggcgctgctgcagatcagcatcccgttcctgctcaagaagctggcccctacctgcaagtcggagctgccccggttccaggagctgatcttcgaggactttgccaggttcatcctggtggaaaacacgtacgaggaggtggtgctgcagaccgtcatgaaggacatcctgcaggctgtgaaggaggccgcggtgcagaggaagcacaacctctaccgggacagcatggtcatgcacaacagcgaccccaacctgcacctgctggccgagggcgcccccatcgactggggcgaggagtacagcaacagcggcgggggcggcagccccagccccagcaccccggagtcagccaccctctcggaaaagcgacggcgcgccaagcaggtggtctctgtggtccaggatgaggaggtggggctgccctttgaggctagccctgagtcaccaccacctgcgtccccggacggtgtcactgagatccgaggcctgctggcccaaggtctgcggcctgagagccccccaccagccggccccctgctcaacggggcccccgctggggagagtccccagcctaaggccgcccccgaggcctcctcgccgcctgcctcacccctccagcatctcctgcctggaaaggctgtggaccttgggccccccaagcccagcgaccaggagactggagagcaggtgtccagccccagcagccaccccgccctccacaccaccaccgaggacagtgcaggggtgcagactgagttctaggccagtgggtccctgactgctgcacatggcacaggccgttcccttccggacccaggcaggctcagctctggggagggcaccctggtctgtgccttgtgggtggaggcggggcagggctgtgtggcaccgccagggagcgggcccacctgagtcactttattgggttcagtcaacactttcttgctccctgttttctcttctgtgggatgatctcagatgcaggggctggttttggggttttcctgcttgtgccaagggctggacactgctggggggctggaaagcccctcccttcctgtccttctgtggcctccatcccctcatgggtgctgccatccttcctggagagagggaggtgaaagctggtgtgagcccagtgggttcccgcccactcacccaggagctggctgggccaggaccgggagagggagcactgctgccctcctggccctgctccttccgcagttaggggtggaccgagcctcgctttccccactgttctggagggaaggggaaggagggggtcttcaggctggagccaggctgggggtgctgggtggagagatgagatttagggggtgcctcatggggtgggcaggcctggggtgaaatgagaaaggcccagaacgtgcaggtctgcggaggggaagtgtcctgagtgaaggaggggaccccatcctggggatgctgggagtgagtgagtgagatggctgagtgagggttatggggagcctgaggttttatgggcctgtgtatccccttctcccggccccagcctgcctccctcctgcccgcctggcccacaggtctccctctggtccctgtccctctggtggttggggatggagcggcagcaaggggtgtaatggggctgggttctgtcttctacaggccaccccgaggtcctcagtggttgcctggggagccggacggggctcctgaggggtacaggttgggtgggccctccctgagggtctggggtcaggctttggcctctgctgcctctcagtcaccaagtcacctccctctgaaaatccagtcccttctttggatgtccttgtgagtcactctgggcctggctgtcgtccctcctcagcttcttgttcctgggacaagggtcaagccaggatgggcccaggcntgggatcccccaccccaggaccccacaggccccctcccctgntgntttgcggggggcagggcagaaatggactccttttgggtccccgaggtggggtcccctcccagccctgcatcctccgtgccctagacctgctccccagaggaggggccttgacccacaggaagtgtggtggcgcctggcaatcagggacccccagctgccgcagccctggtttttggcgcatcttttccctcttgtcccgaagatttgcgcctttagtgccttttgaggggttcccatcatccctccctgatattgtattgaaaatattatgcacactgttcatgcttttactaatcaataaacgctttatttaaaaaaaaaaaaaaaa

SEQ ID NO: 2 - protein, 821 aa, Homo sapiens
MEEVGGRWWGLKPRLAGLRGTGSDCPALAQQQTALKDVLGVRNWASSGPFLLGLSLWRMGWMGGQRSTSLWPWTPGVFSEFLEDGGMGLWFQQEKTGKILTEFLQFYEDQYGVALFNSMRHEIEGTGLPQAQLLWRKVPLDERIVFSGNLFQHQEDSKKWRNRFSLVPHNYGLVLYENKAAYERQVPPRAVINSAGYKILTSVDQYLELIGNSLPGTTAKSGSAPILKCPTQFPLILWHPYARHYYFCMMTEAEQDKWQAVLQDCIRHCNNGIPEDSKVEGPAFTDAIRMYRQSKELYGTWEMLCGNEVQILSNLVMEELGPELKAELGPRLKGKPQERQRQWIQISDAVYHMVYEQAKARFEEVLSKVQQVQPAMQAVIRTDMDQIITSKEHLASKIRAFILPKAEVCVRNHVQPYIPSILEALMVPTSQGFTEVRDVFFKEVTDMNLNVINEGGIDKLGEYMEKLSRLAYHPLKMQSCYEKMESLRLDGLQQRFDVSSTSVFKQRAQIHMREQMDNAVYTFETLLHQELGKGPTKEELCKSIQRVLERVLKKYDYDSSSVRKRFFREALLQISIPFLLKKLAPTCKSELPRFQELIFEDFARFILVENTYEEVVLQTVMKDILQAVKEAAVQRKHNLYRDSMVMHNSDPNLHLLAEGAPIDWGEEYSNSGGGGSPSPSTPESATLSEKRRRAKQVVSVVQDEEVGLPFEASPESPPPASPDGVTEIRGLLAQGLRPESPPPAGPLLNGAPAGESPQPKAAPEASSPPASPLQHLLPGKAVDLGPPKPSDQETGEQVSSPSSHPALHTTTEDSAGVQTEF

SEQ ID NO: 3 - DNA, 4533 bp, Homo sapiens
  CDS (644)..(3019)
  misc_feature (4160)..(4160): n is a, c, g, or t
gggctccctgcacaaatgcgttgggtgatgggggctgaatccagcccacactgcacttgccaagccagctggggccctggcacaagacagtcccagcctgttttcactgactttgctaattctcacggaggcaccatgtggtgtgggaaggcccggtcctcgtaacctctctgctcccaggtccctgaccagtccttaacacacagtggtctttgctcacctgcggcccagctctgggctctccccacagcatcctttgccttgcctccctcccatcttcctctgggccttctctctgctcctgcccaggaaactgtgctctcaggagcgcaggagccagctctcagcccccatctcctgggcactcaccgtactcaggaaatatgttctgaattcaggattatcctcattctactgagaagacctggaggacagaaatcagcaagacctaaaggggagaggaaggagggccaggctggggtggaggtgccccacccgggagcccgggcgcagcctcaccgcaggctgattcacagaaggctcagagggttgcgagggcccaatcggcactgtcatcctgcccaggctctgagtcaccagctggtgaggggcagctgcagcccagcaggaaacaaagtctagcatggaagaggtgggagggaggtggtggggcctgaaaccccgcctggctggccttagaggaactgggagtgactgtccggcactggctcagcagcaaacagctctcaaggacgtgctaggagtcaggaactgggccagctccggtcccttccttttggggctctcactctggaggatggggtggatgggagaaaaaaccgggaagatcctgacggagttcctccagttctatgaagaccagtatggcgtggctctcttcaacagcatgcgccatgagattgagggcacggggctgccgcaggcccagctgctctggcgcaaggtgccactggacgagcgcatcgtcttctcggggaacctcttccagcaccaggaggacagcaagaagtggagaaaccgcttcagcctcgtgccccacaactacgggctggtgctctacgaaaacaaagcggcctatgagcggcaggtcccaccacgagccgtcatcaacagtgcaggctacaaaatcctcacgtccgtggaccaatacctggagctcattggcaactccttaccagggaccacggcaaagtcgggcagtgcccccatcctcaagtgccccacacagttcccgctcatcctctggcatccttatgcgcgtcactactacttctgcatgatgacagaagccgagcaggacaagtggcaggctgtgctgcaggactgcatccggcactgcaacaatggaatccctgaggactccaaggtagagggccctgcgttcacagatgccatccgcatgtaccgacagtccaaggagctgtacggcacctgggagatgctgtgtgggaacgaggtgcagatcctgagcaacctggtgatggaggagctgggccctgagctgaaggcagagctcggcccgcggctgaaggggaaaccgcaggagcggcagcggcagtggatccagatctcggacgccgtgtaccacatggtgtacgagcaggccaaggcgcgcttcgaggaggtgctgtccaaggtgcagcaggtgcagccggccatgcaggccgtcatccgaactgacatggaccaaattatcacctccaaggagcaccttgccagcaagatccgagccttcatcctccccaaggcagaggtgtgcgtgcggaaccatgtccagccctacatcccatccatcctggaggccctgatggtccccaccagccagggcttcactgaggtgcgagatgtcttcttcaaggaggtcacggacatgaacctgaacgtcatcaacgagggcggcattgacaagctgggcgagtacatggagaagctgtcccggctggcgtaccaccccctgaagatgcagagctgctatgagaagatggagtcgctgcgactggacgggctgcagcagcgatttgatgtgtccagcacgtccgtgttcaagcagcgagcccagatccacatgcgggagcaaatggacaatgccgtgtatacgttcgagaccctcctgcaccaggagctggggaaggggcccaccaaggaggagctgtgcaagtccatccagcgggtcctggagcgggtgctgaaaaaatacgactacgacagcagctctgtgcggaagaggttcttccgggaggcgctgctgcagatcagcatcccgttcctgctcaagaagctggcccctacctgcaagtcggagctgccccggttccaggagctgatcttcgaggactttgccaggttcatcctggtggaaaacacgtacgaggaggtggtgctgcagaccgtcatgaaggacatcctgcaggctgtgaaggaggccgcggtgcagaggaagcacaacctctaccgggacagcatggtcatgcacaacagcgaccccaacctgcacctgctggccgagggcgcccccatcgactggggcgaggagtacagcaacagcggcgggggcggcagccccagccccagcaccccggagtcagccaccctctcggaaaagcgacggcgcgccaagcaggtggtctctgtggtccaggatgaggaggtggggctgccctttgaggctagccctgagtcaccaccacctgcgtccccggacggtgtcactgagatccgaggcctgctggcccaaggtctgcggcctgagagccccccaccagccggccccctgctcaacggggcccccgctggggagagtccccagcctaaggccgcccccgaggcctcctcgccgcctgcctcacccctccagcatctcctgcctggaaaggctgtggaccttgggccccccaagcccagcgaccaggagactggagagcaggtgtccagccccagcagccaccccgccctccacaccaccaccgaggacagtgcaggggtgcagactgagttctaggccagtgggtccctgactgctgcacatggcacaggccgttcccttccggacccaggcaggctcagctctggggagggcaccctggtctgtgccttgtgggtggaggcggggcagggctgtgtggcaccgccagggagcgggcccacctgagtcactttattgggttcagtcaacactttcttgctccctgttttctcttctgtgggatgatctcagatgcaggggctggttttggggttttcctgcttgtgccaagggctggacactgctggggggctggaaagcccctcccttcctgtccttctgtggcctccatcccctcatgggtgctgccatccttcctggagagagggaggtgaaagctggtgtgagcccagtgggttcccgcccactcacccaggagctggctgggccaggaccgggagagggagcactgctgccctcctggccctgctccttccgcagttaggggtggaccgagcctcgctttccccactgttctggagggaaggggaaggagggggtcttcaggctggagccaggctgggggtgctgggtggagagatgagatttagggggtgcctcatggggtgggcaggcctggggtgaaatgagaaaggcccagaacgtgcaggtctgcggaggggaagtgtcctgagtgaaggaggggaccccatcctggggatgctgggagtgagtgagtgagatggctgagtgagggttatggggagcctgaggttttatgggcctgtgtatccccttctcccggccccagcctgcctccctcctgcccgcctggcccacaggtctccctctggtccctgtccctctggtggttggggatggagcggcagcaaggggtgtaatggggctgggttctgtcttctacaggccaccccgaggtcctcagtggttgcctggggagccggacggggctcctgaggggtacaggttgggtgggccctccctgagggtctggggtcaggctttggcctctgctgcctctcagtcaccaagtcacctccctctgaaaatccagtcccttctttggatgtccttgtgagtcactctgggcctggctgtcgtccctcctcagcttcttgttcctgggacaagggtcaagccaggatgggcccaggcntgggatcccccaccccaggaccccacaggccccctcccctgntgntttgcggggggcagggcagaaatggactccttttgggtccccgaggtggggtcccctcccagccctgcatcctccgtgccctagacctgctccccagaggaggggccttgacccacaggaagtgtggtggcgcctggcaatcagggacccccagctgccgcagccctggtttttggcgcatcttttccctcttgtcccgaagatttgcgcctttagtgccttttgaggggttcccatcatccctccctgatattgtattgaaaatattatgcacactgttcatgcttttactaatcaataaacgctttatttaaaaaaaaaaaaaaaa

SEQ ID NO: 4 - protein, 791 aa, Homo sapiens
MEEVGGRWWGLKPRLAGLRGTGSDCPALAQQQTALKDVLGVRNWASSGPFLLGLSLWRMGWMGEKTGKILTEFLQFYEDQYGVALFNSMRHEIEGTGLPQAQLLWRKVPLDERIVFSGNLFQHQEDSKKWRNRFSLVPHNYGLVLYENKAAYERQVPPRAVINSAGYKILTSVDQYLELIGNSLPGTTAKSGSAPILKCPTQFPLILWHPYARHYYFCMMTEAEQDKWQAVLQDCIRHCNNGIPEDSKVEGPAFTDAIRMYRQSKELYGTWEMLCGNEVQILSNLVMEELGPELKAELGPRLKGKPQERQRQWIQISDAVYHMVYEQAKARFEEVLSKVQQVQPAMQAVIRTDMDQIITSKEHLASKIRAFILPKAEVCVRNHVQPYIPSILEALMVPTSQGFTEVRDVFFKEVTDMNLNVINEGGIDKLGEYMEKLSRLAYHPLKMQSCYEKMESLRLDGLQQRFDVSSTSVFKQRAQIHMREQMDNAVYTFETLLHQELGKGPTKEELCKSIQRVLERVLKKYDYDSSSVRKRFFREALLQISIPFLLKKLAPTCKSELPRFQELIFEDFARFILVENTYEEVVLQTVMKDILQAVKEAAVQRKHNLYRDSMVMHNSDPNLHLLAEGAPIDWGEEYSNSGGGGSPSPSTPESATLSEKRRRAKQVVSVVQDEEVGLPFEASPESPPPASPDGVTEIRGLLAQGLRPESPPPAGPLLNGAPAGESPQPKAAPEASSPPASPLQHLLPGKAVDLGPPKPSDQETGEQVSSPSSHPALHTTTEDSAGVQTEF